SEQUENCE LISTING (43 sequences)

SEQ ID NO: 1 - DNA, 110 bp, Artificial Sequence
  Description of Artificial Sequence: Synthetic polynucleotide
tgtttgtggtggtgcagatgaacttcagggtttgtctccgggacctgtgcctgccattacaactgtcccggagacaatgaccctgaagttcatctgcaccaccacaaaca

SEQ ID NO: 2 - RNA, 71 nt, Artificial Sequence
  Description of Artificial Sequence: Synthetic oligonucleotide
gggaaacagcgauucaaagaugucauugucuccgggacaguuguaauggcaggcacaggucccggagacaa

SEQ ID NO: 3 - RNA, 31 nt, Artificial Sequence
  Description of Artificial Sequence: Synthetic oligonucleotide
acccugaaguucaucugcaccaccacaaaca

SEQ ID NO: 4 - RNA, 25 nt, Artificial Sequence
  Description of Artificial Sequence: Synthetic oligonucleotide
guggugcagaugaacuucaggguca

SEQ ID NO: 5 - RNA, 75 nt, Artificial Sequence
  Description of Artificial Sequence: Synthetic oligonucleotide
gggaaaucaagaccugugccugccauuacaacugucccggagacaaugacaucuuugaaucgcuguacuacagga

SEQ ID NO: 6 - RNA, 65 nt, Artificial Sequence
  Description of Artificial Sequence: Synthetic oligonucleotide
cagcgauucaaagaugucauugucuccgggacaguuguaauggcaggcacaggucccggagacaa

SEQ ID NO: 7 - RNA, 69 nt, Artificial Sequence
  Description of Artificial Sequence: Synthetic oligonucleotide
ucaagaccugugccugccauuacaacugucccggagacaaugacaucuuugaaucgcuguacuacagga

SEQ ID NO: 8 - RNA, 96 nt, Artificial Sequence
  Description of Artificial Sequence: Synthetic oligonucleotide
acccugaaguuuauuuguaucauugcaaacaacugucccggagacaauuaaacuucaggguaauuauucugguggugcagaugaacuucaggguaa

SEQ ID NO: 9 - RNA, 76 nt, Artificial Sequence
  Description of Artificial Sequence: Synthetic oligonucleotide
cagcgauucaaagaugucauugucuccgaaaggacaguugaaauaauggcagggccauuaaaagcaaugauacaaa

SEQ ID NO: 10 - RNA, 75 nt, Artificial Sequence
  Description of Artificial Sequence: Synthetic oligonucleotide
uguucaucaagaccugugccugccauuacaacugucccggagacaaugacaucuuugaaucgcuguacuacagga

SEQ ID NO: 11 - RNA, 97 nt, Artificial Sequence
  Description of Artificial Sequence: Synthetic oligonucleotide
cacccugaaguuuauuuguaucauugcaaacaacugucccggagacaauuaaacuucaggguaauuauucugguggugcagaugaacuucaggguaa

SEQ ID NO: 12 - RNA, 86 nt, Artificial Sequence
  Description of Artificial Sequence: Synthetic oligonucleotide
uccuguaguacagcgauucaaagaugucauugucuccgaaaggacaguugaaauaauggcagggccauuaaaagcaaugauacaaa

SEQ ID NO: 13 - RNA, 77 nt, Artificial Sequence
  Description of Artificial Sequence: Synthetic oligonucleotide
aagaccugugccugccauuacaacugucccggagacaaugacaucuuugaaucgcuguacuacaggaagauguacgg

SEQ ID NO: 14 - protein, 5 aa, Artificial Sequence
  Description of Artificial Sequence: Synthetic peptide
VVVPP

SEQ ID NO: 15 - DNA, 21 bp, Artificial Sequence
  Description of Artificial Sequence: Synthetic oligonucleotide
  Description of Combined DNA/RNA Molecule: Synthetic oligonucleotide
ggagagagaugggugcgagtt

SEQ ID NO: 16 - DNA, 21 bp, Artificial Sequence
  Description of Artificial Sequence: Synthetic oligonucleotide
  Description of Combined DNA/RNA Molecule: Synthetic oligonucleotide
gaagaaaugaugacagcautt

SEQ ID NO: 17 - DNA, 20 bp, Artificial Sequence
  Description of Artificial Sequence: Synthetic oligonucleotide
  Description of Combined DNA/RNA Molecule: Synthetic oligonucleotide
acaggagcagaugauacamt

SEQ ID NO: 18 - DNA, 21 bp, Artificial Sequence
  Description of Artificial Sequence: Synthetic oligonucleotide
  Description of Combined DNA/RNA Molecule: Synthetic oligonucleotide
cagugcaggggaaagaauatt

SEQ ID NO: 19 - DNA, 21 bp, Artificial Sequence
  Description of Artificial Sequence: Synthetic oligonucleotide
  Description of Combined DNA/RNA Molecule: Synthetic oligonucleotide
gugaaggggcaguaguaautt

SEQ ID NO: 20 - DNA, 21 bp, Artificial Sequence
  Description of Artificial Sequence: Synthetic oligonucleotide
  Description of Combined DNA/RNA Molecule: Synthetic oligonucleotide
auggcaggaagaagcggagtt

SEQ ID NO: 21 - RNA, 151 nt, Artificial Sequence
  Description of Artificial Sequence: Synthetic polynucleotide
uauaaugcaauaaugccacagacaccauuaauuucuuuuaauguuguauuaucuguucuugugucuguggcauuaucgcuuccuuuuaauugccccggaagcggccaucuuccuggccugcauuauauuugugguauuauuguauuauaaa

SEQ ID NO: 22 - RNA, 99 nt, Artificial Sequence
  Description of Artificial Sequence: Synthetic oligonucleotide
aauaaagauaggggggcaauuaaaggaagcucuauuagauacaggagcagaugauacaguauuagaagaaaugaauuugccaggaagauggaaaccaaa

SEQ ID NO: 23 - RNA, 55 nt, Artificial Sequence
  Description of Artificial Sequence: Synthetic oligonucleotide
gaccugugccugccauuacaacugucccggagacaaugacaucuuugaaucgcug

SEQ ID NO: 24 - RNA, 67 nt, Artificial Sequence
  Description of Artificial Sequence: Synthetic oligonucleotide
aagaccugugccugccauuacaacugucccggagacaaugacaucuuugaaucgcuguacuacagga

SEQ ID NO: 25 - RNA, 150 nt, Artificial Sequence
  Description of Artificial Sequence: Synthetic polynucleotide
uauaauguaauaaugccacagacaccauuaauuucucugaaugauguauuaucuguucuugugucuguggcauuaucgcuuccuugaagugccccguacgcggccaucuuccuguccugcauuauauuugugguauuauugvauuauaaa

SEQ ID NO: 26 - RNA, 150 nt, Artificial Sequence
  Description of Artificial Sequence: Synthetic polynucleotide
uauaauggaauaauggcagagacaccauuaauuucuuuuaauguuguauuaucuguucuugugucuguggcauuaucgcuuccuuuaauugccccggaagcggccaucuuccuggccugcauuauauuugugguauuauuguauuauaaa

SEQ ID NO: 27 - RNA, 99 nt, Artificial Sequence; identical to SEQ ID NO: 22
  Description of Artificial Sequence: Synthetic oligonucleotide

SEQ ID NO: 28 - DNA, 96 bp, Artificial Sequence
  Description of Artificial Sequence: Synthetic oligonucleotide
  Description of Combined DNA/RNA Molecule: Synthetic oligonucleotide
gcccacaacaacagugcuauuauuuaaaacaaaucucuaagtcccaacacaacuucacccgaaaaauucuccucuuucacacggacaucacucauu

SEQ ID NO: 29 - RNA, 76 nt, Artificial Sequence
  Description of Artificial Sequence: Synthetic oligonucleotide
caccccaaacaaacaacgcacacuuacuuaauaccaacuugaacaacgggacucccauuacacccaaacacacaaa

SEQ ID NO: 30 - RNA, 95 nt, Artificial Sequence
  Description of Artificial Sequence: Synthetic oligonucleotide
acccugaaguuuauuuguaucauuggaaacaagugucccggagacaauuaaaguucggguaauuauuguggugcugcagaugaacuucaggguaa

SEQ ID NO: 31 - RNA, 96 nt, Artificial Sequence; identical to SEQ ID NO: 8
  Description of Artificial Sequence: Synthetic oligonucleotide

SEQ ID NO: 32 - skipped

SEQ ID NO: 33 - RNA, 76 nt, Artificial Sequence; identical to SEQ ID NO: 9
  Description of Artificial Sequence: Synthetic oligonucleotide

SEQ ID NO: 34 - skipped

SEQ ID NO: 35 - RNA, 65 nt, Artificial Sequence; identical to SEQ ID NO: 6
  Description of Artificial Sequence: Synthetic oligonucleotide

SEQ ID NO: 36 - RNA, 31 nt, Artificial Sequence
  Description of Artificial Sequence: Synthetic oligonucleotide
acccugaaguucaucugcaccacuagaaaca

SEQ ID NO: 37 - RNA, 25 nt, Artificial Sequence
  Description of Artificial Sequence: Synthetic oligonucleotide
guccugcagaugaacuucaggguca

SEQ ID NO: 38 - RNA, 80 nt, Artificial Sequence
  Description of Artificial Sequence: Synthetic oligonucleotide
cagcgauucaaaagauggucauugucuccgaaaaggacaguugaaaauaauggcagggccauuaaaagcaaugauacaag

SEQ ID NO: 39 - RNA, 69 nt, Artificial Sequence
  Description of Artificial Sequence: Synthetic oligonucleotide
aaagaccugugccugccauuacaacugucccggagacagaugacaucuuugaaucgcuguacuacagga

SEQ ID NO: 40 - RNA, 96 nt, Artificial Sequence
  Description of Artificial Sequence: Synthetic oligonucleotide
aaaccugaaguuuauuuguaucauuacaaacaacugucccggagacaauuaaacuucaggguaauuauucuacugaugcagaugaacuucaggaua

SEQ ID NO: 41 - RNA, 144 nt, Artificial Sequence
  Description of Artificial Sequence: Synthetic polynucleotide
cagcgauucaaagaugucauugucuccgaaaaggacaguugaaauaauggcagggccauuaaaagcaaugauacaaaaagaccugugccugccauuacaacugucccggagacaaugacaucuuugaaucgcuguacuacagga

SEQ ID NO: 42 - DNA, 233 bp, Artificial Sequence
  Description of Artificial Sequence: Synthetic oligonucleotide
  Description of Combined DNA/RNA Molecule: Synthetic oligonucleotide
  modified_base (1)..(1): t or u
  modified_base (3)..(5): t or u
  modified_base (7)..(7): t or u
  modified_base (10)..(10): t or u
  modified_base (13)..(13): t or u
  modified_base (19)..(19): t or u
  modified_base (24)..(25): t or u
  modified_base (31)..(33): t or u
  modified_base (35)..(35): t or u
  modified_base (37)..(37): t or u
  modified_base (46)..(46): t or u
  modified_base (48)..(48): t or u
  modified_base (52)..(52): t or u
  modified_base (57)..(58): t or u
  modified_base (64)..(64): t or u
  modified_base (66)..(66): t or u
  modified_base (78)..(78): t or u
  modified_base (84)..(84): t or u
  modified_base (89)..(90): t or u
  modified_base (93)..(93): t or u
  modified_base (95)..(95): t or u
  modified_base (112)..(112): t or u
  modified_base (115)..(115): t or u
  modified_base (121)..(121): t or u
  modified_base (126)..(127): t or u
  modified_base (133)..(133): t or u
  modified_base (144)..(145): t or u
  modified_base (152)..(152): t or u
  modified_base (154)..(154): t or u
  modified_base (157)..(158): t or u
  modified_base (160)..(160): t or u
  modified_base (162)..(162): t or u
  modified_base (172)..(173): t or u
  modified_base (175)..(175): t or u
  modified_base (178)..(178): t or u
  modified_base (191)..(191): t or u
  modified_base (207)..(207): t or u
  modified_base (212)..(213): t or u
  modified_base (216)..(216): t or u
  modified_base (218)..(218): t or u
ngnnngnggnggngcagangaacnncagggnnngncnccgggaccngngccngccannacaacngncccggagacaangacccngaagnncancngcaccaccacaaacagnggngcagangaacnncagggncagcaggcganncaaagangncanngncnccgggacagnngnaanggcaggcacaggncccggagacaaacccngaagnncancngcaccaccacaaaca

SEQ ID NO: 43 - DNA, 75 bp, Artificial Sequence
  Description of Artificial Sequence: Synthetic oligonucleotide
  Description of Combined DNA/RNA Molecule: Synthetic oligonucleotide
  modified_base (1)..(1): t or u
  modified_base (3)..(4): t or u
  modified_base (7)..(7): t or u
  modified_base (15)..(15): t or u
  modified_base (17)..(17): t or u
  modified_base (21)..(21): t or u
  modified_base (26)..(27): t or u
  modified_base (33)..(33): t or u
  modified_base (35)..(35): t or u
  modified_base (47)..(47): t or u
  modified_base (52)..(52): t or u
  modified_base (54)..(56): t or u
  modified_base (60)..(60): t or u
  modified_base (64)..(64): t or u
  modified_base (66)..(66): t or u
  modified_base (69)..(69): t or u
ngnncancaagaccngngccngccannacaacngncccggagacaangacancnnngaancgcngnacnacagga

(19) United States
(12) Patent Application Publication
(10) Pub. No.: US 2015/0240238 A1
(43) Pub. Date: Aug. 27, 2015

(54) THERAPEUTIC RNA SWITCHES COMPOSITIONS AND METHODS OF USE

(71) Applicants: The United of America, as represented by the Secre tary, Department of Health & Human Services, Rockville, MD (US); The Regents of the University of California, Oakland, CA (US)

(72) Inventors: Bruce A. Shapiro, Gaithersburg, MD (US); Eckart H. Bindewald, Frederick, MD (US); Kirill A. Afonin, Frederick, MD (US); Luc Jaeger, Goleta, CA (US); Arti N. Santhanam, Gwynn Oak, MD (US)

(73) Assignee: THE REGENTS OF THE UNIVERSITY OF CALIFORNIA, Oakland, CA (US)

(21) Appl. No.: 14/359,019

(22) PCT Filed: Nov. 19, 2012
(86) PCT No.: PCT/US2012/065932

§ 371 Date: May 16, 2014

Related U.S. Application Data
(60) Provisional application No. 61/561,247, filed on Nov. 17, 2011.
(60) Provisional application No. 61/678,434, filed on Aug. 1, 2012.

(51) Int. Cl. C12N 15/113; A61K 31/713

(52) U.S. Cl. CPC C12N 15/1135; C12N 15/113; A61K 31/713; C12N 15/1131; C12N 2320/30; C12N 2320/31; C12N 2310/11; C12N 2310/14; C12N 2310/151

(57) ABSTRACT

The invention provides for therapeutic RNA switches comprising oligonucleotide sequences complementary to a trigger sequence, an antisense strand oligonucleotide, and a sense strand oligonucleotide complementary to a target nucleic acid molecule.

### CROSS-REFERENCE TO RELATED APPLICATION

This application claims the benefit of U.S. Provisional Application Nos. 61/561,247, filed Nov. 17, 2011, and 61/678,434, filed Aug. 1, 2012, the contents of which are incorporated herein by reference in their entirety.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. The Government has certain rights in this invention.

### BACKGROUND OF THE INVENTION

It is important for drug molecules to evoke a desired cellular effect (such as cell death, up-or down-regulation of a cellular pathway) preferentially in cells that are in a diseased state (targeting). Most drugs employ either passive targeting, cell-surface receptor based targeting or no targeting. For some diseases, such as cancer or viral infections, the correct targeting is a challenge, because the disease state manifests itself mainly through differences in the cell interior, for example in the form of the presence of a certain RNA or protein in the cytoplasm. Improved therapeutic agents that direct targeting to the cells in need of therapy are needed.

### SUMMARY OF THE INVENTION

The present invention provides a new approach to treating diseased cells (cancerous cells or cells infected by a pathogenic agent, e.g., a virus, bacteria, fungus, or parasite), which results in the treatment of the diseased cell through a novel means of selectively inhibiting a target gene in the diseased cell that results in a therapeutic effect. For example, triggering apoptosis or a cell-destructive pathway in those cells which are neoplastic or infected and contain viral nucleic acids. The invention further relates to new compositions of matter, i.e., new forms of RNA molecules called “RNA switches” or “tswRNA” which enable the selective inhibition of target genes in disease cells. In a particular aspect, the invention provides RNA switches which are designed based on certain RNA structure-function relationships and which enable functional cure of a diseased cell via a triggered cell-destruction pathway in the diseased cell. In certain aspects, the therapeutic RNA switches of the invention are RNA molecules that contain several adjacent sequence regions that can bind to a trigger sequence (e.g., a sequence expressed by a target cell, a disease related gene, a cancer related gene, DNA or RNA associated with a pathogenic agent (e.g., a virus, bacteria, fungus, or parasite), such as a viral RNA genome, contained in an infected cell, etc.). In addition, the therapeutic RNA switches contain sequences that represent antisense sequences complementary to one or more known human target genes. In one embodiment of the invention, the sense and antisense sequences that can form an siRNA-like structure are not in any partially formed siRNA-like helices. In other words, the inactive tswRNAs lack any structures that resemble siRNA-like helices. In another embodiment, the one or more human target genes are involved in a disease state such that inhibition of the target gene results in amelioration of the disease state. In other embodiments the one or more human target genes are involved in a cell-destruction pathway, e.g., apoptosis or necrosis. In some embodiments, the antisense sequences are complementary to known human apoptosis inhibitor genes, such as, but not limited to, BCL-2, FLIP, STAT3, and XIAP. The sense and antisense RNAs can be induced to form an siRNA-like hairpin which can inhibit the targeted human gene via the RNA interference (RNAi) pathway. This process is activated through a conformational change occurring in the tswRNA upon binding between the tswRNA and the trigger sequence. For example, in the case of HIV, the therapeutic RNA switch molecule is not in an active conformation when the HIV genome is not present, i.e., when the cell is not infected with HIV. However, when the tswRNA contacts a cell that is infected with virus, the presence of the adjacent sequences in the tswRNA bind to the trigger sequence (i.e. HIV viral RNA) which produces a predetermined conformational change (e.g., by computational design of the molecule) in the molecule. The conformational change reveals a double stranded RNA template, i.e., an siRNA-like hairpin, that is a substrate for the Dicer enzyme which cleaves the RNA. The cleaved RNA releases the siRNA which inhibits the targeted human gene, e.g., a human apoptosis inhibitor gene. Accordingly, when a therapeutic RNA switch of the invention is introduced into a cell that harbors an HIV virus, the therapeutic RNA switch undergoes a conformational change in the presence of the HIV genome which creates an siRNA for an apoptosis inhibitor gene. Processing by the Dicer enzyme releases the siRNA which then inhibits the production of the apoptosis inhibitor gene which results in an increased chance of cell death for the HIV harboring cell. Similarly, in other embodiments, tswRNA are designed wherein the adjacent sequences bind to a trigger sequence that is a disease related gene. Upon binding to the trigger sequence, the tswRNA undergoes a conformational change that reveals a double stranded siRNA-like helix which is processed by Dicer. The liberated siRNA then inhibits a target gene that results in the amelioration of the disease state. In further embodiments the trigger sequence is a sequence that is present in a cell which harbors a nucleic acid or ribonucleic acid molecule that is desirable to inhibit. In some embodiments, delivery of a tswRNA into the cell results in the inhibition of a target gene. In some embodiments, delivery of a tswRNA into the cell results in the treatment/amelioration of infection by a pathogenic agent (e.g., inhibiting a DNA or RNA molecule associated with a virus, bacteria, fungus, or parasite; or inhibiting a target molecule in the cell and causing the infected cell to die).

In one aspect, the invention generally features a therapeutic RNA switch having at least one polynucleotide sequence that can bind to a trigger sequence; and an antisense and sense oligonucleotides in which the antisense oligonucleotide is complementary to a target RNA, where the RNA switch can switch between an inactive state and an active state in the presence of the trigger sequence. In embodiments, no partially formed siRNA-like helices exist in the inactive state. In embodiments, the therapeutic RNA switch undergoes a conformational change in the presence of the trigger sequence which causes the antisense and sense oligonucleotides to form an siRNA-like helix. In embodiments, the siRNA-like molecule reduces or inhibits the target RNA.

In another aspect, the invention features a two-strand therapeutic RNA switch having a complex between an adapter polynucleotide strand and a protofunctional polynucleotide strand, where the adapter polynucleotide can bind a trigger sequence and the protofunctional polynucleotide strand forms an siRNA-like RNA double helix when the adapter polynucleotide strand binds the trigger sequence. In embodiments, the siRNA-like RNA double helix comprises an antisense oligonucleotide and a sense oligonucleotide in which the antisense oligonucleotide is complementary to a target RNA. In embodiments, no partially formed siRNA-like helices exists in the absence of a trigger sequence. In embodiments, the siRNA-like RNA double helix reduces or inhibits the target RNA.

In another aspect, the invention features a four-strand therapeutic RNA/DNA hybrid complex having a complex between a DNA carrier polynucleotide strand, an RNA adapter polynucleotide strand, a sense siRNA strand, and an antisense siRNA strand, where binding of the RNA adapter polynucleotide strand to a trigger sequence removes the RNA adapter strand from the complex and results in a conformational change where the sense siRNA strand and the sense siRNA strand form an siRNA duplex. In embodiments, no partially formed siRNA-like helices exists in the absence of a trigger sequence. In embodiments, the four-strand therapeutic RNA/DNA hybrid complex undergoes a conformational change in the presence of the trigger sequence which causes the antisense and sense oligonucleotides to form an siRNA-like helix. In embodiments, the siRNA-like molecule reduces or inhibits the target RNA.

In any of the above aspects and embodiments, the trigger sequence can be a nucleic acid present in a targeted cell of interest. In embodiments, the nucleic acid is a portion of or is derived from the genome of the targeted cell. For example, the trigger sequence can be an RNA transcript present in a diseased cell or a portion thereof.

In related embodiments, the trigger sequence is a portion of a cancer related gene (e.g., Hif1alpha, VEGF, a DNA repair gene, PARP, miR21, miR7, miR128a, miR210, IL-6, IL-10, JAK, STAT, SMAD, and TNFalpha).

In related embodiments, the nucleic acid is a portion of or is derived from the genome of a pathogenic agent (e.g., the trigger sequence is an RNA transcript derived from the genome of the pathogenic agent or a portion thereof). The pathogenic agent can be a virus, a bacteria, a fungus, or a parasite. In some embodiments, the pathogenic is an RNA virus (e.g., HIV).

In related embodiments, the trigger sequence is an RNA genome of the pathogen or a portion thereof. In embodiments, the RNA genome is the RNA genome of a virus (e.g., HIV).

In any of the above aspects and embodiments, the target RNA is one which produces a therapeutically beneficial result when inhibited.

In embodiments, the target RNA comprises an RNA that encodes a protein involved in a disease process or a portion thereof. In related embodiments, the target RNA comprises an RNA that encodes an apoptosis inhibitor protein or a portion thereof (e.g., Survivin, BCL-2, FLIP, STAT3, and XIAP).

In embodiments, the target RNA is a pathogenic RNA genome, an RNA transcript derived from the genome of the pathogenic agent, or a portion thereof. In some embodiments, the target RNA is a viral RNA genome or a portion thereof (e.g., an HIV genome).

In any of the above aspects and embodiments, the therapeutic strand(s) further comprise a recognition domain that binds a recognition target. The recognition target can be a nucleic acid molecule, a polypeptide, or a fragment thereof. In embodiments, the recognition target is located in or on a target cell. The target cell may be a diseased cell. For example, the diseased cell can be a cancerous cell (a cell having a neoplasia). The diseased cell can also be a cell having a genetic disorder. The disease cell can further be a cell infected with a pathogenic agent (e.g., a virus, a bacteria, a fungus, or a parasite).

In related embodiments, the recognition domain is an aptamer. In embodiments, the aptamer binds a cell membrane polypeptide or cell membrane structure. The cell membrane polypeptide or cell membrane structure can be a disease specific membrane protein or structure (e.g., cancer specific membrane protein or structure, a specific membrane protein or structure associated with infection by a pathogenic agent, and the like). In embodiments, the aptamer binds a molecule in the cell. For example, the aptamer can bind a nucleic acid molecule in the target cell or a portion thereof (e.g., DNA molecule, RNA molecule, or fragment thereof).

The therapeutic strand(s) can also contain functional moieties well known in the art. For example, the therapeutic strand(s) can contain fluorescent tags, domains facilitating cellular uptake, split functionality domains, split lipase, and split GFP. In embodiments, the functional moieties can also be RNA-fluorophore complexes that emit a signal upon association. See Paige, J. S. et al., Science 333:642-646 (2011).

In some embodiments, the therapeutic strands contain at least one of the sequences described in herein (in the description and the figures). For example, the therapeutic strands can contain at least one of the following sequences:

### Construct 4-1 (DNA)

5′-TGTTTGTGGTGGTGCAGATGAACTTCAGGGTTTGTCTCCGGGACCTG
TGCCTGCCATTACAACTGTCCCGGAGACAATGACCCTGAAGTTCATCTGC
ACCACCACAAACA

Adapter 4-1 (RNA, with gggaaa Start Sequence)

5′-gggaaaCAGCGAUUCAAAGAUGUCAUUGUCUCCGGGACAGUUGUAAU
GGCAGGCACAGGUCCCGGAGACAA

EGFP siRNA S1—Sense (RNA)

5′-ACCCUGAAGUUCAUCUGCACCaCcacaaaca

EGFP siRNA S1—Antisense (RNA)

5′guggUGCAGAUGAACUUCAGGGUCA

CTGF mRNA Fragment 4-1 (with gggaaa Start Sequence)

5′-gggaaaUCAAGACCUGUGCCUGCCAUUACAACUGUCCCGGAGACA
AUGACAUCUUUGAAUCGCUGUACUACAGGA

Adapter 4-1b (RNA, without gggaaa Start Sequence)

5′-CAGCGAUUCAAAGAUGUCAUUGUCUCCGGGACAGUUGUAAUGGCAG
GCACAGGUCCCGGAGACAA

CTGF mRNA Fragment 4-1b (without gggaaa Start Sequence)

5′-UCAAGACCUGUGCCUGCCAUUACAACUGUCCCGGAGACAAUGA
CAUCUUUGAAUCGCUGUACUACAGGA

tswRNA Construct-1

5′-ACCCUGAAGUUUAUUUGUAUCAUUGCAAACAACUGUCCCGGAGAC
AAUUAAACUUCAGGGUAAUUAUUCUGGUGGUGCAGAUGAACUUCAGGG
UAA

tswRNA Adapter-1

5′-CAGCGAUUCAAAGAUGUCAUUGUCUCCGAAAGGACAGUUGAAAUAA
UGGCAGGGCCAUUAAAAGCAAUGAUACAAA

CTGF mRNA Fragment-1

5′-UGUUCAUCAAGACCUGUGCCUGCCAUUACAACUGUCCCGGAGACAA
UGACAUCUUUGAAUCGCUGUACUACAGGA

tswRNA Construct-2

5′-CACCCUGAAGUUUAUUUGUAUCAUUGCAAACAACUGUCCCGGAGACA
AUUAAACUUCAGGGUAAUUAUUCUGGUGGUGCAGAUGAACUUCAGGGUAA

tswRNA Adapter-2:

5′-UCCUGUAGUACAGCGAUUCAAAGAUGUCAUUGUCUCCGAAAGGACAG
UUGAAAUAAUGGCAGGGCCAUTUAAAAGCAAUGAYACAAA

CTGF mRNA Fragment-2:

5′-AAGACCUGUGCCUGCCAUUACAACUGUCCCGGAGACAAUGACAUCUU
UGAAUCGCUGUACUACAGGAAGAUGUACGG

In another aspect, the invention features methods for using of the therapeutic molecules described herein.

In aspects, the invention features methods for inhibiting or reducing the expression of a target gene in a cell. In embodiments, the methods involve contacting the cell with a therapeutically effective amount of at least one of the therapeutic molecules described herein. In embodiments, the cell is in a subject.

In aspects, the invention features methods for killing a pathogen infected cell. In embodiments, the methods involve contacting the cell with a therapeutically effective amount of at least one of the therapeutic molecules described herein. In embodiments, the cell is in a subject.

In aspects, the invention features methods for inhibiting replication of a pathogen in a cell. In embodiments, the methods involve contacting the cell with a therapeutically effective amount of at least one of the therapeutic molecules described herein. In embodiments, the cell is in a subject.

In aspects, the invention features methods for reducing pathogenic burden in a subject. In embodiments, the methods involve administering a therapeutically effective amount of a therapeutically effective amount of at least one of the therapeutic molecules described herein. In embodiments, the subject is at risk of developing a pathogenic infection. In embodiments, the subject is diagnosed with having a pathogenic infection.

In aspects, the invention features methods for treating or preventing a pathogenic infection in a subject. In embodiments, the methods involve administering a therapeutically effective amount of a therapeutically effective amount of at least one of the therapeutic molecules described herein. In embodiments, the methods reduce the pathogenic burden, thereby treating or preventing the pathogenic infection. In embodiments, the methods induce death in infected cell, thereby treating or preventing the pathogenic infection.

In any of the above aspects and embodiments, the subject can be a mammal (e.g., human).

In any of the above aspects and embodiments, the pathogen can be a virus, bacteria, fungus, or parasite. In some embodiments, the pathogen is a virus (e.g., HIV).

In any of the above aspects and embodiments, the methods can involve further contacting the cell with a therapeutically effective amount of a second therapeutic agent or administering a therapeutically effective amount of the second therapeutic agent to the subject. The second therapeutic agent can treat the pathogenic infection or the symptoms associated with pathogenic infection. For example, the second therapeutic agent can be an anti-viral agent, an anti-bacterial agent, an anti-fungal agent, or an anti-parasitic agent. Such agents are well known in the art, and it is within the purview of a physician to select and determine the appropriate dosage of the second therapeutic agent. See, e.g., Drug Information Handbook: A Comprehensive Resource for All Clinicians and Healthcare Professionals, 20^{th} Ed., C. F. Lacy et al. (eds.) (Lexi-Comp 2011); Johns Hopkins ABX Guide: Diagnosis & Treatment of Infectious Diseases, 2^{nd} Ed., J. G. Bartlett et al. (eds.) (Jones & Bartlett Publishers 2010); and Mandell, Douglas, and Bennett's Principles and Practice of Infectious Diseases: Expert Consult Premium Edition, 7^{th} Ed., G. L. Mandell (ed.) (Churchill Livingstone 2009); The Sanford Guide to Antimicrobial Therapy 2012, 42^{nd} Ed., D. N. Gilbert et al. (eds.) (Antimicrobial Therapy 2012); Clinical Infectious Disease 2013, 11^{th} Ed., C. G. Weber (ed.) (Pacific Primary Care Software 2012), the contents of which are hereby incorporated by reference in their entirety.

In aspects, the invention features methods for killing a neoplastic cell. In embodiments, the methods involve contacting the cell with a therapeutically effective amount of at least one of the therapeutic molecules described herein. In embodiments, the cell is in a subject.

In aspects, the invention features methods for treating a subject having a neoplasia. In embodiments, the methods involve administering a therapeutically effective amount of a therapeutically effective amount of at least one of the therapeutic molecules described herein.

In embodiments, the neoplastic cell is a cancer cell which is present in a solid tumor. In embodiments, the cancer is selected from the group consisting of breast cancer, prostate cancer, melanoma, glioblastomas, colon cancer, ovarian cancer, and non-small cell lung cancer.

In related embodiments, the methods involve contacting the cell with a therapeutically effective amount of a second therapeutic agent or administering a therapeutically effective amount of the second therapeutic agent to the subject. In some embodiments, the second therapeutic agent is an anti-cancer agent. Anti-cancer agents are well known in the art, and any such agent is suitable for use in the present invention. See, e.g., Anticancer Drugs: Design, Delivery and Pharmacology (Cancer Etiology, Diagnosis and Treatments) (eds. Spencer, P. & Holt, W.) (Nova Science Publishers, 2011); Clinical Guide to Antineoplastic Therapy: A Chemotherapy Handbook (ed. Gullatte) (Oncology Nursing Society, 2007); Chemotherapy and Biotherapy Guidelines and Recommendations for Practice (eds. Polovich, M. et al.) (Oncology Nursing Society, 2009); Physicians' Cancer Chemotherapy Drug Manual 2012 (eds. Chu, E. & DeVita, Jr., V. T.) (Jones & Bartlett Learning, 2011); DeVita, Hellman, and Rosenberg's Cancer: Principles and Practice of Oncology (eds. DeVita, Jr., V. T. et al.) (Lippincott Williams & Wilkins, 2011); and Clinical Radiation Oncology (eds. Gunderson, L. L. & Tepper, J. E.) (Saunders) (2011), the contents of which are hereby incorporated by references in their entirety. For example, nonlimiting examples of anti-cancer agents include Abiraterone Acetate, Afatinib, Aldesleukin, Alemtuzumab, Alitretinoin, Altretamine, Amifostine, Aminoglutethimide Anagrelide, Anastrozole, Arsenic Trioxide, Asparaginase, Azacitidine, Azathioprine, Bendamustine, Bevacizumab, Bexarotine, Bicalutamide, Bleomycin, Bortezomib, Busulfan, Capecitabine, Carboplatin, Carmustine, Cetuximab, Chlorambucil, Cisplatin, Cladribine, Crizotinib, Cyclophosphamide, Cytarabine, Dacarbazine, Dactinomycin, Dasatinib, Daunorubicin, Denileukin diftitox, Decitabine, Docetaxel, Dexamethasone, Doxifluridine, Doxorubicin, Epirubicin, Epoetin Alpha, Epothilone, Erlotinib, Estramustine, Etinostat, Etoposide, Everolimus, Exemestane, Filgrastim, Floxuridine, Fludarabine, Fluorouracil, Fluoxymesterone, Flutamide, folate linked alkaloids, Gefitinib, Gemcitabine, Gemtuzumab ozogamicin, GM-CT-01, Goserelin, Hexamethylmelamine, Hydroxyureas, Ibritumomab, Idarubicin, Ifosfamide, Imatinib, Interferon alpha, Interferon beta, Irinotecan, Ixabepilone, Lapatinib, Leucovorin, Leuprolide, Lenalidomide, Letrozole, Lomustine, Mechlorethamine, Megestrol, Melphalan, Mercaptopurine, Methotrexate, Mitomycin, Mitoxantrone, Nelarabine, Nilotinib, Nilutamide, Octreotide, Ofatumumab, Oprelvekin, Oxaliplatin, Paclitaxel, Panitumumab, Pemetrexed, Pentostatin, polysaccharide galectin inhibitors, Procarbazine, Raloxifene, Retinoic acids, Rituximab, Romiplostim, Sargramostim, Sorafenib, Streptozocin, Sunitinib, Tamoxifen, Temsirolimus, Temozolamide, Teniposide, Thalidomide, Thioguanine, Thiotepa, Tioguanine, Topotecan, Toremifene, Tositumomab, Trametinib, Trastuzumab, Tretinoin, Valrubicin, VEGF inhibitors and traps, Vinblastine, Vincristine, Vindesine, Vinorelbine, Vintafolide (EC145), Vorinostat, or a salt thereof.

In any of the above aspects and embodiments, the pathogen can be any known virus, bacteria, fungus, or parasite known in the art. See, e.g., Clinical Infectious Disease 2013, 11^{th} Ed., C. G. Weber (ed.) (Pacific Primary Care Software 2012).

Exemplary bacterial pathogens include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Lactobacillus, Legionella, Leptospira, Listeria, Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

Exemplary viruses include, but are not limited to, Retroviridae (e.g., human immunodeficiency viruses, such as HIV-1 (also referred to as HDTV-III, LAVE or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronoviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g. influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of pathogenic fungi include, without limitation, Alternaria, Aspergillus, Basidiobolus, Bipolaris, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, and Candida lusitaniae, Coccidioides, Cladophialophora, Cryptococcus, Cunninghamella, Curvularia, Exophiala, Fonsecaea, Histoplasma, Madurella, Malassezia, Plastomyces, Rhodotorula, Scedosporium, Scopulariopsis, Sporobolomyces, Tinea, and Trichosporon.

Parasites can be classified based on whether they are intracellular or extracellular. An “intracellular parasite” as used herein is a parasite whose entire life cycle is intracellular. Examples of human intracellular parasites include Leishmania, Plasmodium, Trypanosoma cruzi, Toxoplasma gondii, Babesia, and Trichinella spiralis. An “extracellular parasite” as used herein is a parasite whose entire life cycle is extracellular. Extracellular parasites capable of infecting humans include Entamoeba histolytica, Giardia lamblia, Enterocytozoon bieneusi, Naegleria and Acanthamoeba as well as most helminths. Yet another class of parasites is defined as being mainly extracellular but with an obligate intracellular existence at a critical stage in their life cycles. Such parasites are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora, Cryptosporidium, Eimeria, Neospora, Sarcocystis, and Schistosoma. In one aspect, the invention relates to the prevention and treatment of infection resulting from intracellular parasites and obligate intracellular parasites which have at least in one stage of their life cycle that is intracellular. In some embodiments, the invention is directed to the prevention of infection from obligate intracellular parasites which are predominantly intracellular. An exemplary and non-limiting list of parasites for some aspects of the invention include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii. Blood-borne and/or tissues parasites include Plasmodium, Babesia microti, Babesia divergens, Leishmania tropica, Leishmania, Leishmania braziliensis, Leishmania donovani, Trypanosoma gambiense and Trypanosoma rhodesiense (African sleeping sickness), Trypanosoma cruzi (Chagas' disease), and Toxoplasma gondii.

The invention also features compositions (including pharmaceutical compositions) containing at least one of the therapeutic molecules described herein. In embodiments, the composition contains a pharmaceutically acceptable excipient, carrier, or diluent.

In embodiments, the compositions are used for one of at least one of the methods described herein.

In embodiments, the compositions further contain at least one additional therapeutic agent. In some embodiments, the second therapeutic agent treats or reduces the symptoms associated with infection by a pathogenic agent. In some embodiments, the second therapeutic agent is an anti-cancer agent.

The invention further features kits containing the therapeutic molecules and/or compositions described herein. In embodiments, the kits are used for at least one of the methods described herein. In related embodiments, the kits further contain instructions for using the kits in at least one of the methods described herein.

In some embodiments, the kits contain at least one additional therapeutic agent. In embodiments, the second therapeutic agent treats or reduces the symptoms associated with infection by a pathogenic agent. In embodiments, the second therapeutic agent is an anti-cancer agent.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “therapeutic RNA switch” or “tswRNA” is meant an RNA molecule or RNA containing molecular complex that has at least one region that is complementary to a trigger sequence (e.g., the recognition domain). The trigger sequence can be any sequence (either RNA or DNA) that is present in a targeted cell (e.g., a gene specific for a target cell, a disease related gene, a cancer related gene, a DNA or RNA molecule associated with a pathogenic agent, etc.). Also present in the cell is a target RNA, the inhibition of which would result in amelioration of a disease state (e.g., an mRNA of a target gene, RNA intermediate of a pathogenic agent (e.g., bacterial mRNA), RNA genome of a pathogenic agent (e.g., RNA virus), and the like). As shown in FIG. 1, the tswRNA contains i) at least one antisense sequence (that is complementary to the target RNA (referred to in FIG. 1 as the siRNA guide strand), and ii) a complementary sense sequence for each of the antisense sequence(s) (referred to in FIG. 1 as the siRNA passenger strand). The tswRNA recognition domain can be the same as, contiguous to, adjacent to, or unrelated to the trigger sequence. In the absence of binding to the trigger sequence, the tswRNA is in an inactive state wherein the tswRNA sense and tswRNA antisense regions do not form an siRNA-like molecule. Upon binding to the trigger sequence, the tswRNA undergoes a conformational change that causes the tswRNA sense and tswRNA antisense regions to form an siRNA-like molecule which can be processed by Dicer. Cleavage by Dicer releases the tswRNA antisense strand, thereby initiating targeted RNA cleavage.

By “target gene,” “target RNA,” or “target human RNA” is meant an RNA that encodes a polypeptide that has a functionality whose inhibition is desired. For example, a target gene is a disease related gene the inhibition of which results in the amelioration of the disease state.

By “trigger sequence” is meant a sequence (RNA or DNA) that is present in a targeted cell that binds to sequences in a tswRNA which cause the tswRNA to switch from an inactive to an active state. In certain embodiments the trigger sequence is a disease related sequence or a sequence that is specific for a disease state.

By “agent” is meant any small molecule chemical compound, antibody, nucleic acid molecule, or polypeptide, or fragments thereof, and may include the tswRNAs of the invention.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Non-limiting examples of diseases include cancer, infection by a pathogenic agent (e.g., virus, bacteria, fungus, or parasite), and the like. For example, the disease can be, but is not limited to, viral infections, RNA virus infections, HIV, AIDS, breast cancer, prostate cancer, glioblastoma, osteosarcoma, colon cancer, non-small cell lung cancer, ovarian cancer, and melanoma

By “effective amount” is meant the amount of a required agent to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen of the agent. Such amount is referred to as an “effective” amount.

The invention provides a number of targets that are useful for the development of highly specific drugs to treat a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

As used herein, “antisense strand” refers to a single stranded nucleic acid molecule which has a sequence complementary to that of a target RNA. When the antisense strand contains modified nucleotides with base analogs, it is not necessarily complementary over its entire length, but must at least hybridize with a target RNA.

As used herein, “sense strand” refers to a single stranded nucleic acid molecule which has a sequence complementary to that of an antisense strand. When the antisense strand contains modified nucleotides with base analogs, the sense strand need not be complementary over the entire length of the antisense strand, but must at least duplex with the antisense strand.

As used herein, “guide strand” also termed “antisense strand” refers to a single stranded nucleic acid molecule of a dsRNA or dsRNA-containing molecule (e.g., a processed tswRNA of the invention), which has a sequence sufficiently complementary to that of a target RNA to result in RNA interference. After cleavage of the dsRNA or dsRNA-containing molecule by Dicer, a fragment of the guide strand remains associated with RISC (RNA-induced silencing complex), binds a target RNA as a component of the RISC complex, and promotes cleavage of a target RNA by RISC. As used herein, the guide strand does not necessarily refer to a continuous single stranded nucleic acid and may comprise a discontinuity, optionally at a site that is cleaved by Dicer. A guide strand can be an antisense strand.

As used herein, “passenger strand” refers to an oligonucleotide strand of a dsRNA or dsRNA-containing molecule, which has a sequence that is complementary to that of the guide strand. As used herein, the passenger strand does not necessarily refer to a continuous single stranded nucleic acid and may comprise a discontinuity, optionally at a site that is cleaved by Dicer. A passenger strand can be a sense strand.

By “complementarity” is meant that a nucleic acid can form hydrogen bond(s) with another nucleic acid sequence by either traditional Watson-Crick or other non-traditional types.

In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence. In one embodiment, a dsRNA molecule of a formulation of the invention comprises about 19 to about 30 (e.g., about 19, 20, 21, 22, 23, 24, 25, 26, 27, 28, 29, or 30 or more) nucleotides that are complementary to one or more target nucleic acid molecules or a portion thereof.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

The following Detailed Description, given by way of example, but not intended to limit the invention to specific embodiments described, may be understood in conjunction with the accompanying drawings, in which:

FIG. 1 is an embodiment of the invention depicting a schematic of the pathway of the therapeutic switching RNAs described herein. In Step 1, due to the absence of a target viral genome, the tswRNA is in an inactive conformation. In Step 2, when in the presence of viral genome, the tswRNA binds to the viral genome and undergoes a conformational change to form an active tswRNA, wherein the active form comprises an exposed or revealed siRNA-like portion. In Step 3, Dicer recognizes and cleave the siRNA-like portion of the active tswRNA. In Step 4, the guide strand portion of the tswRNA is incorporated into RISC (RNA induced silencing complex) forming a loaded RISC complex. In Step 5, the targeted human mRNA is recognized by the loaded RISC complex and is degraded.

FIG. 2 is a schematic of the predicted secondary structure of an illustrative example of a tswRNA.

FIGS. 3A and 3B depicts the predicted 3D structures of an unbound (inactive state) tswRNA.

FIG. 4 depicts the predicted structure of a bound (active state) tswRNA.

FIG. 5 shows the sequences of an illustrative example of a 2-strand tswRNA.

FIG. 6 is an illustration of the predicted folding of 2-strand tswRNA protofunctional strand in the absence of a trigger sequence.

FIG. 7 is an illustration of the secondary structure model of an illustrative example 2-strand tswRNA in an inactive conformation.

FIG. 8 is an illustration of the predicted structure of 2-strand tswRNA complex without the adapter strand, i.e., in an active conformation in the presence of a trigger sequence.

FIG. 9 shows the predicted structure of an illustrative example of a 2-strand tswRNA including pseudoknot interaction.

FIG. 10 is an illustration showing the conformational changes of a 2-strand tswRNA in the transition from the inactive form to the active form in the presence of trigger sequence (CTGF mRNA).

FIG. 11 is an illustration showing the structure of a 2-strand tswRNA.

FIG. 12 is an illustration of a 3D model of 2-strand tswRNA complex.

FIG. 13 is an illustration of a 4-strand RNA/DNA hybrid complex comprised of a carrier strand, an adapter strand, an antisense siRNA strand, and a sense siRNA strand.

FIG. 14 is an illustration showing the structural features of a 4-strand RNA/DNA hybrid complex and the conformational changes in response to a trigger sequence.

FIG. 15 shows the sequences of an illustrative example of a 4-strand RNA/DNA hybrid complex.

FIG. 16 is the predicted secondary structure of the siRNA strands of an illustrative 4-strand RNA/DNA hybrid complex.

FIG. 17 is an illustration showing the conformational changes of a 4-strand RNA/DNA hybrid complex in the transition from the inactive form to the active form in the presence of trigger sequence (CTGF mRNA).

FIG. 18 shows the assembly of a four-stranded switch in a sequential matter. First, the adapter-construct-antisense trimer was formed and then sense strand was added and incubated at 55° C. for 20 minutes. When all four strands (adapter, construct, antisense and sense) are mixed together, the formation of four-stranded switch is impossible (last lane on the right). Three different protocols were tested for trimer formation. Protocols #1: apdaptor, construct and toehold mixed together and incubated at 95° C. for 2 minutes and then snap-cooled to 55° C. and incubated for 20 minutes. Protocols #2: apdaptor, construct and toehold mixed together and incubated at 95° C. for 2 minutes and then snap-cooled to room temperature (RT) and incubated for 20 minutes. Protocols #3: apdaptor, construct and toehold mixed together and incubated at 95° C. for 2 minutes and then snap-cooled to 20° C. and incubated for 20 minutes.

### DETAILED DESCRIPTION OF THE INVENTION

The invention features compositions and methods that are useful for specifically inhibiting a target nucleic acid molecule in a diseased cell. In certain embodiments the diseased cell is a neoplastic cell or a cell infected with a pathogenic agent, and inhibition of the target nucleic acid molecule results in the death and eradication of the diseased cell and/or the pathogenic agent. The invention features a therapeutic RNA switch (tswRNA) which is an RNA molecule or an RNA containing molecular complex that has at least one sequence that is complementary to a trigger sequence (e.g., a gene specific for a target cell, a disease related gene, a cancer related gene, a DNA or RNA molecule associated with a pathogenic agent, etc.) and has an antisense sequence that is complementary to the target nucleic acid molecule (e.g., a target human RNA, such as a target RNA encoded by a gene of interest, the inhibition of which will tend to lead to the treatment of the diseased cell, e.g., death via an apoptosis or necrosis pathway; a pathogenic RNA, the inhibition of which will inhibit replication of the infectious agent and thereby treat the infection and/or its associated symptoms). The tswRNA also contains a sense strand that is complementary to the antisense sequence (tswRNA sense strand). In the absence of a trigger sequence, the tswRNA is in an inactive state, in which case the tswRNA sense and tswRNA antisense sequences do not form an siRNA-like molecule. However, in the presence of a trigger sequence the tswRNA undergoes a conformational change as a result of binding to the trigger sequence. The conformational change causes the tswRNA to be in an active state where the tswRNA sense and tswRNA antisense sequences form an siRNA-like molecule. The siRNA-like molecule is processed by Dicer, after which the tswRNA antisense strand (i.e., the guide strand) becomes part of the RISC complex which results in the degradation of the target RNA.

In contrast to previously described concepts of siRNA-containing RNA constructs that undergo a conformational change upon binding to a trigger molecule, the claimed constructs contain in their inactive state no partially formed siRNA-like helices. In the inactive conformation the sequence regions corresponding to siRNA guide (tswRNA antisense) and siRNA passenger (tswRNA antisense) are not interacting via RNA base pairing. Instead, an siRNA-like helix is formed only if the construct is bound to a trigger sequence. This overcomes two problems of RNAi-activating RNA switches. First, the undesired processing by Dicer of the RNA switch in its inactive state is improbable. Secondly, partially degraded RNA switches (that occur due to nuclease degradation) are less likely to lead to the formation of an siRNA-like helix, and are thus less likely to inadvertently activate the RNA-interference pathway. This has the effect that the described RNA switches will have fewer side effects because the therapeutic RNA-interference pathway is only activated by the RNA switch if it is intact and it is in its active conformation. In addition, in embodiments, the active conformation can be designed to contain a minimal number of single stranded nucleotides thus minimizing the effects of nucleases.

In embodiments, a tswRNA of the invention comprises nucleic acid strands at least one of them consisting completely or partially of RNA (2-strand tswRNA). The adapter strand comprises RNA, DNA, or other nucleotide analogs and binds to a protofunctional RNA strand, in an inactive conformation. In the presence of a trigger sequence, the tswRNA binds to the trigger sequence. The remaining unbound protofunctional strand undergoes a conformational change that forms an siRNA-like RNA double-helix. The siRNA-like double-helix is recognized and processed by DICER, thus leading to the activation of the RNA silencing pathway. The activation of the RNA silencing pathway leads to the down-regulation of the desired target nucleic acid molecule or pathway.

In yet another embodiment, an RNA/DNA hybrid complex is provided comprising four strands: a DNA carrier strand, an RNA adapter strand, a sense siRNA strand, and an antisense siRNA strand. The four strands bind to form a hybrid complex. In the absence of a trigger sequence the RNA/DNA hybrid complex is in an inactive form. In the presence of a trigger sequence, the tswRNA binds to the trigger sequence which removes the adapter strand from the RNA/DNA hybrid complex. The remaining complex comprises the carrier strand, the sense siRNA strand, and the antisense siRNA strand which undergoes a conformational change that leads to the formation of an siRNA duplex and a self-folding carrier strand. The majority of the adapter strand is reverse-complementary to the trigger sequence. The carrier strand comprises several regions: a region that can bind to the sense siRNA, a region that can bind to the adapter strand, and a region that can bind to the antisense siRNA. The carrier strand further comprises an additional complementarity region that promotes the formation of the siRNA duplex after removal of the adapter strand. The siRNA duplex is recognized and processed by DICER, thus leading to the activation of the RNA silencing pathway. The activation of the RNA silencing pathway leads to the down-regulation of the desired target nucleic acid molecule or pathway.

In an embodiment, the trigger sequence is a sequence that is present in a target cell whereby the tswRNA is activated in the target cell. In another embodiment the target cell is a diseased cell and the trigger sequence is a disease related or disease specific sequence (e.g., disease marker). In other embodiments, the trigger sequence is a pathogenic DNA or RNA molecule. Activation of the tswRNA results in inhibition of a target RNA (e.g., cellular RNA or pathogenic RNA) and thereby ameliorates the disease state.

In one aspect of the invention, the diseased cell is a cell infected with a pathogenic agent (e.g., virus, bacteria, fungus, or parasite). In embodiments, the tswRNA targets a cellular protein, thereby inducing the infected cell to die. In other embodiments, the tswRNA targets genomic RNA (e.g., viral RNA) or an RNA intermediate (e.g., bacterial mRNA) associated with the pathogen. Degradation of the pathogenic RNA results in reduction of pathogen load, which thereby treats the infection and/or associated symptoms. In related embodiments, the trigger sequence can be a pathogenic DNA or RNA, or a cellular DNA or RNA (e.g., cell marker) associated with pathogenic infection.

In embodiments, the pathogen is a virus and the trigger sequence is a viral genome. In related embodiments, the virus is HIV. In some related embodiments, the viral genome is an HIV genome and the target RNAs encode apoptosis inhibitor proteins. When such a tswRNA is introduced into an HIV infected cell the tswRNA results in the inhibition of the apoptosis inhibitory molecules which causes the HIV infected cell to die.

The invention also provides methods of treating a pathogenic infection in a subject by administering an effective amount of a tswRNA. In some embodiments, tswRNA is activated in the presence of pathogenic RNA or DNA and results in the destruction of targeted apoptosis inhibitors, thereby resulting in the death of the infected cells.

In embodiments, the pathogen is a virus (e.g., RNA virus). In related embodiments, tswRNA is activated in the presence of the HIV genome and results in the destruction of targeted apoptosis inhibitors, thereby resulting in the death of the HIV infected cells and treatment and/or reduction of the infection in the subject.

In another embodiment, the diseased cell is a neoplastic cell. In related embodiments, the trigger sequence is a cancer related gene. When such a tswRNA is introduced into a neoplastic cell the tswRNA activates and reveals an siRNA-like helix. The siRNA produced by the activation inhibits a target gene that results in the death of the neoplastic cell.

The invention also provides methods of treating subjects having neoplasia by administering an effective amount of a tswRNA. In embodiments, the tswRNA is activated in the presence of cancer related genes and results in the destruction of the targeted apoptosis inhibitors, thereby resulting in the death of the neoplastic cells and treatment and/or reduction of neoplasia from the subject.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which target genes are implicated.

In one embodiment, the invention provides a method of monitoring treatment progress. The method includes the step of determining a level of diagnostic marker for diseased cells, (e.g., any target delineated herein modulated by a compound herein, a protein or indicator thereof, etc.) or diagnostic measurement (e.g., screen, assay) in a subject suffering from or susceptible to a disorder or symptoms thereof associated with the diseased cells, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Pharmaceutical Therapeutics

The present disclosure provides tswRNAs that decrease the expression or activity of target proteins in diseased cells. In one embodiment, the disclosure provides pharmaceutical compositions comprising a tswRNA that inhibits the expression or activity of an apoptosis inhibitor in the diseased cell (e.g., neoplastic cell or pathogen infected cell). In a further embodiment, the diseased cell is infected with an HIV virus. For therapeutic uses, the compositions or agents identified using the methods disclosed herein may be administered systemically, for example, formulated in a pharmaceutically-acceptable carrier or delivery vehicle. Preferable routes of administration include, for example, subcutaneous, intravenous, interperitoneally, intramuscular, or intradermal injections that provide continuous, sustained levels of the drug in the patient. Treatment of human patients or other animals can be carried out using a therapeutically effective amount of a cancer therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and the clinical symptoms of cancer progression or metastasis. Generally, amounts can be in the range of those used for other agents used in the treatment of cancer progression or metastasis, although in certain instances lower amounts will be needed because of the increased specificity of the compound. A compound can be administered at a dosage that controls the clinical or physiological symptoms of cancer progression or metastasis as determined by a diagnostic method known to one skilled in the art, or using any that assay that measures the transcriptional activation of a gene associated with cancer progression or metastasis.

### Formulation of Pharmaceutical Compositions

The administration of a tswRNA of the disclosure or analog thereof for the treatment of a diseased cell may be by any suitable means that results in a concentration of the tswRNA that, combined with other components, is effective in ameliorating, reducing, eradicating, or stabilizing the disease. Preferably, the mode of delivery or administration tends to result in the entry of the tswRNA in a cell, and preferably, a diseased cell wherein the tswRNA may interact with a trigger sequence specific for the diseased cell. In one embodiment, administration of the tswRNA reduces the expression or activity of a target nucleic acid molecule, the inhibition of which results in amelioration of the disease. In another embodiment, the tswRNA is administered to a subject for the prevention or treatment of a disease associated with neoplasia or pathogenic infection.

Methods of administering such tswRNA are known in the art. The disclosure provides for the therapeutic administration of an agent by any means known in the art. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). Suitable formulations include forms for oral administration, depot formulations, formulations for delivery by a patch, and semi-solid dosage forms to be topically or trans-dermally delivered.

Pharmaceutical compositions according to the disclosure may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (saw-tooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target tumor cells by using carriers or chemical derivatives to deliver the therapeutic agent to a particular cell type whose function is perturbed in cancer. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

The delivery vehicles contemplated by the invention that may carry the therapeutic tswRNAs to the cells of the infected subject may also be targeted to particular cells by employment of any suitable targeting means. Such means may include incorporating a delivery moiety or targeting moiety into the delivery vehicle to enable the targeted delivery of the tswRNA to specified cells or tissues or area of the body.

As used herein, the term “delivery moiety” or “targeting moiety” is a moiety that is capable of enhancing the ability of an associated or attached delivery vehicle or naked RNA of the invention to associate with, bind, or enter a cell, cell of a tissue or subject, cell type, tissue or location within a subject, either in vitro or in vivo. In certain embodiments, delivery moieties are polypeptides, carbohydrates or lipids. Optionally, delivery moieties are aptamers, antibodies, antibody fragments or nanobodies. Exemplary delivery moieties include tumor targeting moieties, such as somatostatin (sst2), bombesin/GRP, luteinizing hormone-releasing hormone (LHRH), neuropeptide Y (NPY/Y1), neurotensin (NT1), vasoactive intestinal polypeptide (VIP/VPAC1) and cholecystokinin (CCK/CCK2). In certain embodiments, a delivery moiety is non-covalently associated with a compound of the invention. In other embodiments, a delivery moiety is attached to a delivery vehicle of the invention, and is optionally covalently attached. In further embodiments, a delivery moiety is attached to a delivery vehicle of the invention, and is optionally covalently attached. In additional embodiments, a delivery moiety is attached directly to a “cargo” of the invention (e.g., a tswRNA of the invention), optionally covalently. In particular embodiments the oligonucleotides of the invention are delivered as a “naked” RNA attached to an aptamer wherein the aptamer targets a particular cell type.

In certain instances, the formulations of the invention comprise a ligand, such as a targeting ligand that may interact with a specific receptor on a target cell type. Exemplary ligands include lipids, amphipathic lipids, carrier compounds, bioaffinity compounds, biomaterials, biopolymers, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

In certain other embodiments, the delivery vehicles carrying the tswRNA cargo to virus-infected cells may include lipid-based carrier systems suitable for use in the present invention, including lipoplexes (see, e.g., U.S. Patent Publication No. 20030203865; and Zhang et al., J. Control Release, 100:165-180 (2004)), pH-sensitive lipoplexes (see, e.g., U.S. Patent Publication No. 2002/0192275), reversibly masked lipoplexes (see, e.g., U.S. Patent Publication Nos. 2003/0180950), cationic lipid-based compositions (see, e.g., U.S. Pat. No. 6,756,054; and U.S. Patent Publication No. 2005/0234232), cationic liposomes (see, e.g., U.S. Patent Publication Nos. 2003/0229040, 2002/0160038, and 2002/0012998; U.S. Pat. No. 5,908,635; and PCT Publication No. WO 01/72283), anionic liposomes (see, e.g., U.S. Patent Publication No. 2003/0026831), pH-sensitive liposomes (see, e.g., U.S. Patent Publication No. 2002/0192274; and AU 2003/210303), antibody-coated liposomes (see, e.g., U.S. Patent Publication No. 2003/0108597; and PCT Publication No. WO 00/50008), cell-type specific liposomes (see, e.g., U.S. Patent Publication No. 2003/0198664), liposomes containing nucleic acid and peptides (see, e.g., U.S. Pat. No. 6,207,456), liposomes containing lipids derivatized with releasable hydrophilic polymers (see, e.g., U.S. Patent Publication No. 2003/0031704), lipid-entrapped nucleic acid (see, e.g., PCT Publication Nos. WO 03/057190 and WO 03/059322), lipid-encapsulated nucleic acid (see, e.g., U.S. Patent Publication No. 2003/0129221; and U.S. Pat. No. 5,756,122), other liposomal compositions (see, e.g., U.S. Patent Publication Nos. 2003/0035829 and 2003/0072794; and U.S. Pat. No. 6,200,599), stabilized mixtures of liposomes and emulsions (see, e.g., EP1304160), emulsion compositions (see, e.g., U.S. Pat. No. 6,747,014), and nucleic acid micro-emulsions (see, e.g., U.S. Patent Publication No. 2005/0037086), the disclosures of which are each incoporated in their entireties by reference.

The delivery vehicles used to administer the tswRNAs of the invention also may include polymer-based carrier systems which may include, but are not limited to, cationic polymer-nucleic acid complexes (i.e., polyplexes). To form a polyplex, cargo (e.g., a tswRNA of the invention) is typically complexed with a cationic polymer having a linear, branched, star, or dendritic polymeric structure that condenses the cargo into positively charged particles capable of interacting with anionic proteoglycans at the cell surface and entering cells by endocytosis. In some embodiments, the polyplex comprises nucleic acid (e.g., tswRNAs) complexed with a cationic polymer such as polyethylenimine (PEI) (see, e.g., U.S. Pat. No. 6,013,240; commercially available from Qbiogene, Inc. (Carlsbad, Calif.) as In vivo jetPEI®, a linear form of PEI), polypropylenimine (PPI), polyvinylpyrrolidone (PVP), poly-L-lysine (PLL), diethylaminoethyl (DEAE)-dextran, poly(β-amino ester) (PAE) polymers (see, e.g., Lynn et al., J. Am. Chem. Soc., 123:8155-8156 (2001)), chitosan, polyamidoamine (PAMAM) dendrimers (see, e.g., Kukowska-Latallo et al., Proc. Natl. Acad. Sci. USA, 93:4897-4902 (1996)), porphyrin (see, e.g., U.S. Pat. No. 6,620,805), polyvinylether (see, e.g., U.S. Patent Publication No. 20040156909), polycyclic amidinium (see, e.g., U.S. Patent Publication No. 20030220289), other polymers comprising primary amine, imine, guanidine, and/or imidazole groups (see, e.g., U.S. Pat. No. 6,013,240; PCT Publication No. WO/9602655; PCT Publication No. WO95/21931; Zhang et al., J. Control Release, 100:165-180 (2004); and Tiera et al., Curr. Gene Ther., 6:59-71 (2006)), and a mixture thereof. In other embodiments, the polyplex comprises cationic polymer-nucleic acid complexes as described in U.S. Patent Publication Nos. 2006/0211643, 2005/0222064, 2003/0125281, and 2003/0185890, and PCT Publication No. WO 03/066069; biodegradable poly(β-amino ester) polymer-nucleic acid complexes as described in U.S. Patent Publication No. 2004/0071654; microparticles containing polymeric matrices as described in U.S. Patent Publication No. 2004/0142475; other microparticle compositions as described in U.S. Patent Publication No. 2003/0157030; condensed nucleic acid complexes as described in U.S. Patent Publication No. 2005/0123600; and nanocapsule and microcapsule compositions as described in AU 2002358514 and PCT Publication No. WO 02/096551. These disclosures are incorporated herein by reference.

In certain instances, the tswRNA cargo may be complexed with cyclodextrin or a polymer thereof. Non-limiting examples of cyclodextrin-based carrier systems include the cyclodextrin-modified polymer-nucleic acid complexes described in U.S. Patent Publication No. 2004/0087024; the linear cyclodextrin copolymer-nucleic acid complexes described in U.S. Pat. Nos. 6,509,323, 6,884,789, and 7,091,192; and the cyclodextrin polymer-complexing agent-nucleic acid complexes described in U.S. Pat. No. 7,018,609. In certain other instances, the cargo (e.g., a nucleic acid such as a DsiRNA) may be complexed with a peptide or polypeptide. An example of a protein-based carrier system includes, but is not limited to, the cationic oligopeptide-nucleic acid complex described in PCT Publication No. WO95/21931. These disclosures are incorporated herein by reference.

### Pharmaceutical Compositions

In certain embodiments, the present invention provides for a pharmaceutical composition comprising a tswRNA of the present invention. Such compositions can be suitably formulated and introduced into the environment of the cell by any means that allows for a sufficient portion of the inventive compositions to enter the cell to deliver a cargo/payload. Many formulations are known in the art and can be used so long as the inventive formulation gains entry to the target cells so that it can act. See, e.g., U.S. published patent application Nos. 2004/0203145 A1 and 2005/0054598 A1. For example, the inventive formulation of the instant invention can be further formulated in buffer solutions such as phosphate buffered saline solutions and capsids. Cationic lipids, such as lipofectin (U.S. Pat. No. 5,705,188), cationic glycerol derivatives, and polycationic molecules, such as polylysine (published PCT International Application WO 97/30731), can be used within the formulations of the instant invention. Optionally, Oligofectamine, Lipofectamine (Life Technologies), NC388 (Ribozyme Pharmaceuticals, Inc., Boulder, Colo.), or FuGene 6 (Roche) may be employed, all of which can be used according to the manufacturer's instructions.

Such compositions can include the lipidic formulation and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular cargo delivery has also been discussed in Straubringer et al., Methods Enzymol., 101: 512; Mannino et al, Biotechniques, 6: 682; Nicolau et al., Crit. Rev. Ther. Drug Carrier Syst., 6:239 (1989); and Behr, Ace. Chem. Res., 26: 274. Still other methods of administering lipid-based therapeutics are described in, for example, U.S. Pat. Nos. 3,993,754; 4,145,410; 4,235,871; 4,224,179; 4,522,803; and 4,588,578. The lipid-cargo formulation particles can be administered by direct injection at the site of disease or by injection at a site distal from the site of disease (see, e.g., Culver, HUMAN GENE THERAPY, MaryAnn Liebert, Inc., Publishers, New York. pp. 70-71). The formulations of the present invention, either alone or in combination with other suitable components, can be made into aerosols (i.e., they can be “nebulized”) to be administered via inhalation (e.g., intranasally or intratracheally; see, Brigham et al., Am. J. Sci., 298: 278). Aerosol formulations can be placed into pressurized acceptable propellants, such as dichlorodifluoromethane, propane, nitrogen, and the like.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) is also well-known in the pharmaceutical arts. Similarly, transmucosal drug delivery in the form of a polytetrafluoroetheylene support matrix is described in U.S. Pat. No. 5,780,045.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, formulations can be administered, for example, by intravenous infusion, orally, topically, intraperitoneally, intravesically, or intrathecally.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, optional methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the formulations are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active formulations are formulated into ointments, salves, gels, or creams as generally known in the art.

The formulations can also be prepared in the form of suppositories (e.g., with conventional suppository bases such as cocoa butter and other glycerides) or retention enemas for rectal delivery.

The formulations can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

In certain embodiments, the formulations can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In certain aspects, the formulations are prepared with carriers that will protect the formulations against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Formulations suitable for oral administration can consist of, e.g.: (a) liquid solutions, such as an effective amount of the packaged cargo (e.g., nucleic acid) suspended in diluents such as water, saline, or PEG 400; (b) capsules, sachets, or tablets, each containing a predetermined amount of the cargo, as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the cargo in a flavor, e.g., sucrose, as well as pastilles comprising the cargo in an inert base, such as gelatin and glycerin or sucrose and acacia emulsions, gels, and the like containing, in addition to the cargo, carriers known in the art.

The methods of the present invention may be practiced in a variety of hosts. Exemplary hosts include mammalian species, such as primates (e.g., humans and chimpanzees as well as other nonhuman primates), canines, felines, equines, bovines, ovines, caprines, rodents (e.g., rats and mice), lagomorphs, and swine.

Toxicity and therapeutic efficacy of such formulations can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Formulations which exhibit high therapeutic indices can be preferred. While formulations that exhibit toxic side effects may be used, care should be taken to design a delivery system that targets such formulations to the site of affected tissue in order to minimize potential damage to uninfected cells and, thereby, reduce side effects.

The data obtained from the cell culture assays and animal studies can be used in formulating a range of dosage for use in humans. The dosage of such formulations optionally lies within a range of circulating concentrations that include the ED₅₀ with little or no toxicity. The dosage may vary within this range depending upon the dosage form employed and the route of administration utilized. For any formulation used in a method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of formulation (i.e., an effective dosage) depends on the formulation selected. For instance, if a tswRNA formulation is selected, single dose amounts (of either the formulation as a whole or of a cargo component of such formulation) in the range of approximately 1 pg to 1000 mg may be administered; in some embodiments, 10, 30, 100, or 1000 pg, or 10, 30, 100, or 1000 ng, or 10, 30, 100, or 1000 μg, or 10, 30, 100, or 1000 mg may be administered. In some embodiments, 1-5 g of the formulations can be administered. The formulations can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, nucleic acid or antibody can include a single treatment or, optionally, can include a series of treatments.

It can be appreciated that the method of introducing formulations into the environment of the cell will depend on the type of cell and the make up of its environment. For example, when the cells are found within a liquid, one optional formulation is with a lipid formulation such as in lipofectamine and the formulations can be added directly to the liquid environment of the cells. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering peptides, proteins and nucleic acids (e.g., oligonucleotides) are known and can be used. For suitable methods of introducing dsRNA (e.g., tswRNA agents), see U.S. published patent application No. 2004/0203145 A1.

Suitable amounts of a formulation must be introduced and these amounts can be empirically determined using standard methods. Typically, effective concentrations of individual formulations, or of individual cargoes of a formulation, in the environment of a cell will be about 50 nanomolar or less, 10 nanomolar or less, or compositions in which concentrations of about 1 nanomolar or less can be used. In another embodiment, methods utilizing a concentration of about 200 picomolar or less, and even a concentration of about 50 picomolar or less, about 20 picomolar or less, about 10 picomolar or less, or about 5 picomolar or less can be used in many circumstances.

Suitably formulated pharmaceutical compositions of this invention can be administered by any means known in the art such as by parenteral routes, including intravenous, intramuscular, intraperitoneal, subcutaneous, transdermal, airway (aerosol), rectal, vaginal and topical (including buccal and sublingual) administration. In some embodiments, the pharmaceutical compositions are administered by intravenous or intraparenteral infusion or injection.

### Parenteral Compositions

In preferred embodiments, the pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active therapeutic(s), the composition may include suitable parenterally acceptable carriers and/or excipients. The active therapeutic(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the disclosure may be in the form suitable for sterile injection. To prepare such a composition, the suitable active therapeutic(s) are dissolved or suspended in a parenterally acceptable liquid vehicle.

### Controlled Release Parenteral Compositions

Controlled release parenteral compositions may be in the form of suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices. Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Inhibitory Nucleic Acids

The tswRNA molecules described herein operate by forming inhibitory nucleic acid molecules in the presence of trigger sequences. Such inhibitory nucleic acids include single and double stranded nucleic acid molecules (e.g., DNA, RNA, and analogs thereof) that bind a nucleic acid molecule that encodes target RNA (e.g., antisense oligonucleotide molecules, siRNA, shRNA, and the like) as well as nucleic acid molecules that bind directly to a target polypeptide to modulate its biological activity (e.g., aptamers).

### Ribozymes

Catalytic RNA molecules or ribozymes that include an antisense target RNA sequence of the present disclosure can be used to inhibit expression of target RNAs in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the disclosure also features a catalytic RNA molecule that includes, in the binding arm, an antisense RNA having between eight and nineteen consecutive nucleobases. In preferred embodiments of this disclosure, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the disclosure and those skilled in the art will recognize that all that is important in an enzymatic nucleic acid molecule of this disclosure is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39, 2002). Given the sequence of a target gene, siRNAs may be designed to inactivate that gene. Such siRNAs, for example, could be administered directly to an affected tissue, or administered systemically. The nucleic acid sequence of an Parl gene can be used to design small interfering RNAs (siRNAs). The 21 to 25 nucleotide siRNAs may be used, for example, as therapeutics to treat cancer progression or metastasis.

The inhibitory nucleic acid molecules of the present disclosure may be employed as double-stranded RNAs for RNA interference (RNAi)-mediated knock-down of target RNA expression. In one embodiment, target RNA expression is reduced in a virus infected cell. In another embodiment, the target RNA encodes apoptosis inhibitor proteins and the cells are infected with HIV. RNAi is a method for decreasing the cellular expression of specific proteins of interest (reviewed in Tuschl, Chem Bio Chem 2:239-245, 2001; Sharp, Gene Dev 15:485-490, 2000; Hutvagner and Zamore, Curr Opin Genet Devel 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the disclosure, a double-stranded RNA (dsRNA) molecule is made that includes between eight and nineteen consecutive nucleobases of a nucleobase oligomer of the disclosure. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Gene Dev 16:948-958, 2002. Paul et al. Nat Biotechnol 20:505-508, 2002; Sui et al. Proc Natl Acad Sci USA 99:5515-5520, 2002; Yu et al. Proc Natl Acad Sci USA 99:6047-6052, 2002; Miyagishi et al. Nat Biotechnol 20:497-500, 2002; and Lee et al. Nat Biotechnol 20:500-505, 2002, each of which is hereby incorporated by reference.

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

The invention also contemplates certain modifications that can be made to the tswRNAs of the invention that serve to stabilize and/or enhance the function of the molecules, so long as the modification does not prevent the tswRNAs from serving as a substrate for Dicer. It was previously found that base paired deoxyribonucleotides can be attached to DsiRNA molecules, resulting in enhanced RNAi efficacy and duration, provided that such extension is performed in a region of the extended molecule that does not interfere with Dicer processing (e.g., 3′ of the Dicer cleavage site of the sense strand/5′ of the Dicer cleavage site of the antisense strand). In one embodiment, one or more modifications are made that enhance Dicer processing of the tswRNA cargo. In a second embodiment, one or more modifications are made that result in more effective RNAi generation. In a third embodiment, one or more modifications are made that support a greater RNAi effect. In a fourth embodiment, one or more modifications are made that result in greater potency per each tswRNA cargo molecule to be delivered to the cell. Modifications can be incorporated in the 3′-terminal region, the 5′-terminal region, in both the 3′-terminal and 5′-terminal region or in some instances in various positions within the sequence. With the restrictions noted above in mind, any number and combination of modifications can be incorporated into the tswRNA cargo. Where multiple modifications are present, they may be the same or different. Modifications to bases, sugar moieties, the phosphate backbone, and their combinations are contemplated. Either 5′-terminus can be phosphorylated.

Examples of modifications contemplated for the phosphate backbone include phosphonates, including methylphosphonate, phosphorothioate, and phosphotriester modifications such as alkylphosphotriesters, and the like. Examples of modifications contemplated for the sugar moiety include 2′-alkyl pyrimidine, such as 2′-O-methyl, 2′-fluoro, amino, and deoxy modifications and the like (see, e.g., Amarzguioui et al., 2003). Examples of modifications contemplated for the base groups include abasic sugars, 2-O-alkyl modified pyrimidines, 4-thiouracil, 5-bromouracil, 5-iodouracil, and 5-(3-aminoallyl)-uracil and the like. Locked nucleic acids, or LNA's, could also be incorporated. Many other modifications are known and can be used so long as the above criteria are satisfied. Examples of modifications are also disclosed in U.S. Pat. Nos. 5,684,143, 5,858,988 and 6,291,438 and in U.S. published patent application No. 2004/0203145 A1. Other modifications are disclosed in Herdewijn (2000), Eckstein (2000), Rusckowski et al. (2000), Stein et al. (2001); Vorobjev et al. (2001). Each are incorporated herein by reference.

The invention encompasses stabilized oligonucleotides having modifications that protect against 3′ and 5′ exonucleases as well as endonucleases. Such modifications desirably maintain target affinity while increasing stability in vivo. In various embodiments, oligonucleotides of the invention include chemical substitutions at the ribose and/or phosphate and/or base positions of a given nucleobase sequence. For example, oligonucleotides of the invention include chemical modifications at the 2′ position of the ribose moiety, circularization of the aptamer, 3′ capping and ‘spiegelmer’ technology. Oligonucleotides having A and G nucleotides sequentially replaced with their 2′-OCH3 modified counterparts are particularly useful in the methods of the invention. Such modifications are typically well tolerated in terms of retaining affinity and specificity. In various embodiments, oligonucleotides include at least 10%, 25%, 50%, or 75% modified nucleotides. In other embodiments, as many as 80-90% of the olignucleotides' nucleotides contain stabilizing substitutions. In other embodiments, 2′-OMe containing oligonucleotides are synthesized. Such oligonucleotides are desirable because they are inexpensive to synthesize and natural polymerases do not accept 2′-OMe nucleotide triphosphates as substrates so that 2′-OMe nucleotides cannot be recycled into host DNA. Using methods described herein, oligonucleotides will be selected for increased in vivo stability. In one embodiment, oligonucleotides having 2′-F and 2′-OCH₃ modifications are used to generate nuclease resistant aptamers. In other embodiments, the nucleic acids of the invention have one or more locked nucleic acids (LNA). LNA refers to a modified RNA nucleotide. The ribose of the LNA is modified with an extra bridge connecting the 2′ oxygen and the 4′ carbon which locks the ribose into the North or 3′-endo conformation. See e.g., Kaur, H. et al., Biochemistry, vol. 45, pages 7347-55; and Koshkin, A. A., et al., Tetrahedron, vol. 54, pages 3607-3630. In other embodiments, one or more oligonucleotides of the invention incorporate a morpolino structure where the nucleic acid bases are bound to morpholine rings instead of deoxyribose rings and are linked through phosphorodiamidate groups instead of phosphates. See eg., Summerton, J. and Weller, D., Antisense & Nucleic Acid Drug Development, vol. 7, pages 187-195. Yet other modifications, include (PS)-phosphate sulfur modifications wherein the phosphate backbone of the nucleic acid is modified by the substitution of one or more sulfur groups for oxygen groups in the phosphate backbone. Other modifications that stabilize nucleic acids are known in the art and are described, for example, in U.S. Pat. No. 5,580,737; and in U.S. Patent Application Publication Nos. 20050037394, 20040253679, 20040197804, and 20040180360.

### Delivery of Nucleotide-Base Oligomers

The present invention also contemplates the delivery and/or administration of naked inhibitory nucleic acid molecules of the invention (e.g., the tswRNAs of the invention), or analogs thereof, which are capable of entering mammalian cells and inhibiting expression of a gene of interest, and in particular, where the mammalian cell is infected with a target RNA. Nonetheless, it may be desirable to utilize a formulation that aids in the delivery of the tswRNAs of the invention, or any nucleic acids of the invention, to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Detection of Delivered Nucleic Acids

In embodiments that utilize lipid-based delivery vehicles to administer the tswRNAs of the invention, the cargo-lipid formulation particles can be detected in the subject at about 8, 12, 24, 48, 60, 72, or 96 hours, or 6, 8, 10, 12, 14, 16, 18, 19, 22, 24, 25, or 28 days after administration of the particles. The presence of the particles can be detected in the cells, tissues, or other biological samples from the subject. The particles may be detected, e.g., by direct detection of the particles; detection of the modified cargo (e.g., nucleic acid); where the cargo is a nucleic acid, detection of a nucleic acid that silences expression of a target sequence; detection of the target and/or target sequence of interest (i.e., by detecting expression or reduced expression of the target and/or sequence of interest), or a combination thereof. A cargo-lipid formulation comprising a peptide-modified lipid of the invention, when compared to a control formulation, results in at least 5%, 10%, 20%, 30%, 40%, 50%, 60%, 70%, 80%, 90%, 95%, or even 100% increase in the detection of cargo-lipid formulation particles, as measured by a detection method, e.g., fluorescent tag or PCR.

Cargo-lipid formulation particles can be detected using any methods known in the art. For example, a label can be coupled directly or indirectly to a component of the carrier system using methods well-known in the art. A wide variety of labels can be used, with the choice of label depending on sensitivity required, ease of conjugation with the carrier system component, stability requirements, and available instrumentation and disposal provisions. Suitable labels include, but are not limited to, spectral labels such as fluorescent dyes (e.g., fluorescein and derivatives, such as fluorescein isothiocyanate (FITC) and Oregon Green™; rhodamine and derivatives such Texas red, tetrarhodimine isothiocynate (TRITC), etc., digoxigenin, biotin, phycoerythrin, AMCA, CyDyes™, and the like; radiolabels such as ³H, ¹²⁵I, ³⁵S, 'C, ³²P, ³³P, etc.; enzymes such as horseradish peroxidase, alkaline phosphatase, etc.; spectral colorimetric labels such as colloidal gold or colored glass or plastic beads such as polystyrene, polypropylene, latex, etc. The label can be detected using any means known in the art.

Cargoes can be detected and quantified herein by any of a number of means well-known to those of skill in the art. The detection of nucleic acids proceeds by well-known methods such as Southern analysis, Northern analysis, gel electrophoresis, PCR, radiolabeling, scintillation counting, and affinity chromatography. Additional analytic biochemical methods such as spectrophotometry, radiography, electrophoresis, capillary electrophoresis, high performance liquid chromatography (HPLC), thin layer chromatography (TLC), and hyperdiffusion chromatography may also be employed for a cargo of a formulation of the invention.

For nucleic acid cargoes, the selection of a nucleic acid hybridization format is not critical. A variety of nucleic acid hybridization formats are known to those skilled in the art. For example, common formats include sandwich assays and competition or displacement assays. Hybridization techniques are generally described in, e.g., “Nucleic Acid Hybridization, A Practical Approach,” Eds. Hames and Higgins, IRL Press (1985).

Sensitivity of a hybridization assays may be enhanced through use of a nucleic acid amplification system which multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR) the ligase chain reaction (LCR), Qβ-replicase amplification and other RNA polymerase mediated techniques (e.g., NASBA™) are found in Sambrook et al, In Molecular Cloning: A Laboratory Manual, Cold Spring Harbor Laboratory Press (2000); and Ausubel et al, SHORT PROTOCOLS IN MOLECULAR BIOLOGY, eds., Current Protocols, Greene Publishing Associates, Inc. and John Wiley & Sons, Inc. (2002); as well as U.S. Pat. No. 4,683,202; PCR Protocols, A Guide to Methods and Applications (Innis et al. eds.) Academic Press Inc. San Diego, Calif. (1990); Arnheim & Levinson (Oct. 1, 1990), C&EN 36; The Journal Of NIH Research, 3:81 (1991); Kwoh et al., Proc. Natl. Acad. ScL USA, 86:1173 (1989); Guatelli et al., Proc. Natl. Acad. Sci. USA, 87: 1874 (1990); Lomell et al., J. Clin. Chem., 35: 1826 (1989); Landegren et al, Science, 241:1077 (1988); Van Brunt, Biotechnology, 8:291 (1990); Wu and Wallace, Gene, 4:560 (1989); Barringer et al, Gene, 89: 117 (1990); and Sooknanan and Malek, Biotechnology, 13:563 (1995). Improved methods of cloning in vitro amplified nucleic acids are described in U.S. Pat. No. 5,426,039. Other methods described in the art are the nucleic acid sequence based amplification (NASBA™, Cangene, Mississauga, Ontario) and Qβ-replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

Nucleic acids for use as probes, e.g., in vitro amplification methods, for use as gene probes, or as inhibitor components are typically synthesized chemically according to the solid phase phosphoramidite triester method described by Beaucage et al, Tetrahedron Letts., 22: 1859 1862 (1981), e.g., using an automated synthesizer, as described in Needham VanDevanter et al, Nucleic Acids Res., 12:6159 (1984). Purification of polynucleotides, where necessary, is typically performed by either native acrylamide gel electrophoresis or by anion exchange HPLC as described in Pearson et al, J. Chrom., 255: 137 149 (1983). The sequence of the synthetic polynucleotides can be verified using the chemical degradation method of Maxam and Gilbert (1980) in Grossman and Moldave (eds.) Academic Press, New York, Methods in Enzymology, 65:499.

An alternative means for determining the level of transcription of a nucleic acid/gene (e.g., target gene) is in situ hybridization. In situ hybridization assays are well-known and are generally described in Angerer et al., Methods Enzymol, 152: 649. In an in situ hybridization assay, cells are fixed to a solid support, typically a glass slide. If DNA is to be probed, the cells are denatured with heat or alkali. The cells are then contacted with a hybridization solution at a moderate temperature to permit annealing of specific probes that are labeled. The probes are optionally labeled with radioisotopes or fluorescent reporters.

### Dosage

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### Therapeutic Methods

The present disclosure provides methods of treating diseases (for example, neoplasia, pathogenic infections, etc.), particularly by specifically inhibiting or reducing target nucleic acid molecules in diseased cells. The methods comprise administering a therapeutically effective amount of a pharmaceutical composition comprising a nucleic acid that contains at least one sequence that is complementary to a trigger sequence and sense and antisense sequences wherein the antisense (i.e., guide strand) is complementary to a target RNA wherein the sense and antisense sequences form an siRNA-like molecule in the presence of the trigger sequence. Thus the invention provides for the treatment of any disease where the inhibition of a target gene results in the treatment of the cell or amelioration of a disease state. One embodiment is a method of treating a subject suffering from or susceptible to virus (HIV) infection. Another embodiment is a method of treating a subject suffering from or susceptible to neoplasia. The method includes the step of administering to the subject a therapeutic amount or an amount of a compound herein sufficient to treat the disease or symptom thereof, under conditions such that the disease is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the disclosure, which include prophylactic treatment, in general comprise administration of a therapeutically effective amount of the agent herein, such as a compound of the formulae herein to a subject (e.g., animal, human) in need thereof, including a mammal, particularly a human. Such treatment will be suitably administered to subjects, particularly humans, suffering from, having, susceptible to, or at risk for a cancer progression or metastasis or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The agent herein may be also used in the treatment of any other disorders in which transcriptional activity may be implicated.

In one embodiment, the disclosure provides a method of monitoring treatment progress. The method includes the step of determining a level of diagnostic marker (Marker) (e.g., a marker indicative of HIV infection) or diagnostic measurement (e.g., screen, assay) in a subject suffering from or susceptible to a disorder or symptoms thereof associated with HIV or AIDS, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In one embodiment, the Marker is the HIV virus itself. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this disclosure; this pre-treatment level of Marker can then be compared to the level of Marker in the subject after the treatment commences, to determine the efficacy of the treatment.

### Kits

The disclosure provides kits for the treatment or prevention of a disease. Certain embodiments provide for kits for the treatment or prevention of a viral infection including HIV infection or AIDS. Another embodiment provides for kits for the treatment or prevention of neoplasia. In one embodiment, the kit includes a therapeutic or prophylactic composition containing an effective amount of an agent of the invention (e.g., tswRNA) in unit dosage form. In some embodiments, the kit comprises a sterile container which contains a therapeutic or prophylactic compound; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired an agent of the disclosure is provided together with instructions for administering it to a subject having or at risk of developing HIV infection or AIDS. The instructions will generally include information about the use of the composition for the treatment or prevention of HIV infection or AIDS. In other embodiments, the instructions include at least one of the following: description of the compound; dosage schedule and administration for treatment or prevention of HIV infection or AIDS or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In addition, if desired an agent of the disclosure is provided together with instructions for administering it to a subject having or at risk of developing neoplasia. The instructions will generally include information about the use of the composition for the treatment or prevention of neoplasia. In other embodiments, the instructions include at least one of the following: description of the compound; dosage schedule and administration for treatment or prevention of neoplasia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In another embodiment, a composition of the disclosure has activity that inhibits the protein FLIP. In a further embodiment, the composition is given in combination with drugs that are or resemble death ligands such as “Fas/CD95-ligang, and TRAIL.” In such an embodiment the compositions of the disclosure sensitizes a tumor cell to apoptosis by the combination drug.

### Combination Therapies for the Treatment of Disease

Compositions and methods of the disclosure may be used in combination with any conventional therapy known in the art. In one embodiment, a composition of the disclosure (e.g., a composition comprising a tswRNA) having anti-HIV activity may be used in combination with any anti-viral known in the art.

In another embodiment a composition of the disclosure having anti-neoplastic activity may be used in combination with one or more chemotherapeutic agents. In other embodiments the one or more chemotherapeutics is selected from the group consisting of abiraterone acetate, altretamine, anhydrovinblastine, auristatin, azacitidine, bendamustine, bevacizumab, bexarotene, bicalutamide, BMS-184476, 2,3,4,5,6-pentafluoro-N-(3-fluoro-4-methoxyphenyl)benzene sulfonamide, bleomycin, bortezomib, N,N-dimethyl-L-valyl-L-valyl-N-methyl-L-valyl-L-prolyl-L-proline-t-butylamide, cachectin, capecitabine, cemadotin, cetuximab, chlorambucil, cyclophosphamide, 3′,4′-didehydro-4′-deoxy-8′-norvin-caleukoblastine, docetaxol, doxetaxel, carboplatin, carmustine (BCNU), cisplatin, cryptophycin, cytarabine, dacarbazine (DTIC), dactinomycin, dasatinib, daunorubicin, dolastatin, doxorubicin (adriamycin), erlotinib, etoposide, 5-fluorouracil, finasteride, flutamide, hydroxyurea and hydroxyurea taxanes, ifosfamide, imatinib, irinotecan, lenalidomide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, panitumumab, pazopanib, prednimustine, procarbazine, rituximab, RPR109881, sorafenib, estramustine phosphate, sunitinib, tamoxifen, tasonermin, taxol, temozolomide, trastuzumab, tretinoin, vinblastine, vincristine, vindesine sulfate, vinflunine, and vorinostat.

### Recombinant Polypeptide Expression

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

The present invention is described by reference to the following Examples, which are offered by way of illustration and are not intended to limit the invention in any manner. Standard techniques well known in the art or the techniques specifically described below were utilized.

### Example 1

### Therapeutic RNA Switch Targeting Infected or Neoplastic Cells

Computationally designed therapeutic RNA switches represent an important step towards a functional cure of infection and cancer. Using small interfering RNAs (siRNAs) or small hairpin RNAs (shRNAs), it is routinely possible to knock down target mRNA expression. Furthermore, it is possible to induce cell death (apoptosis) if one simultaneously targets several human apoptosis inhibitor genes with siRNAs. The key idea of this proposal is to design siRNA analogs that will induce apoptosis only when triggered by the presence of the pathogenic DNA or RNA or cancer DNA and RNA markers in the cytoplasm.

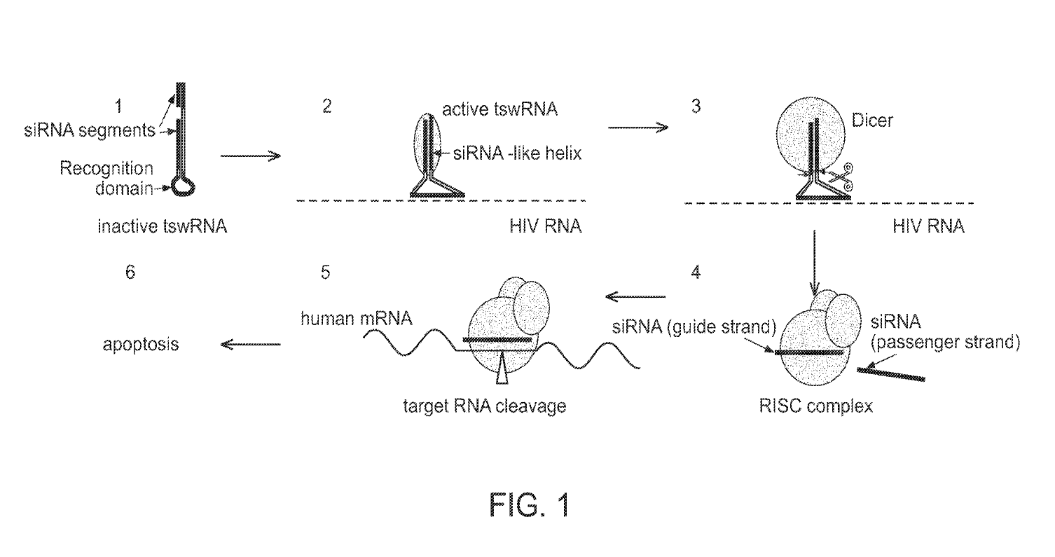

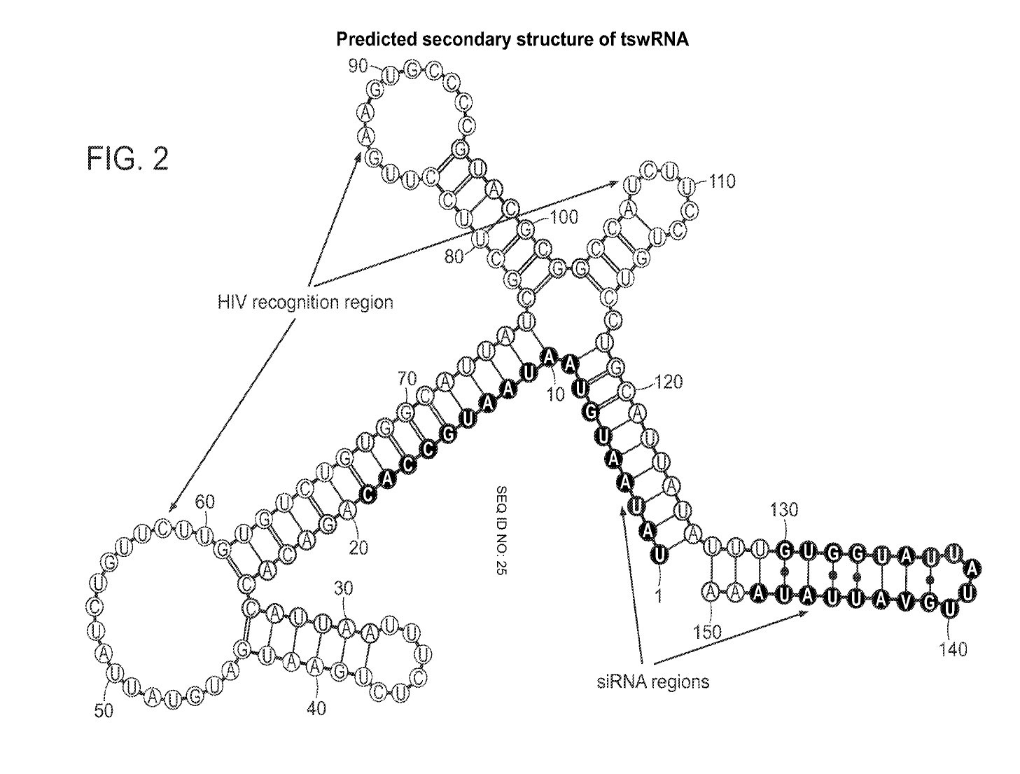

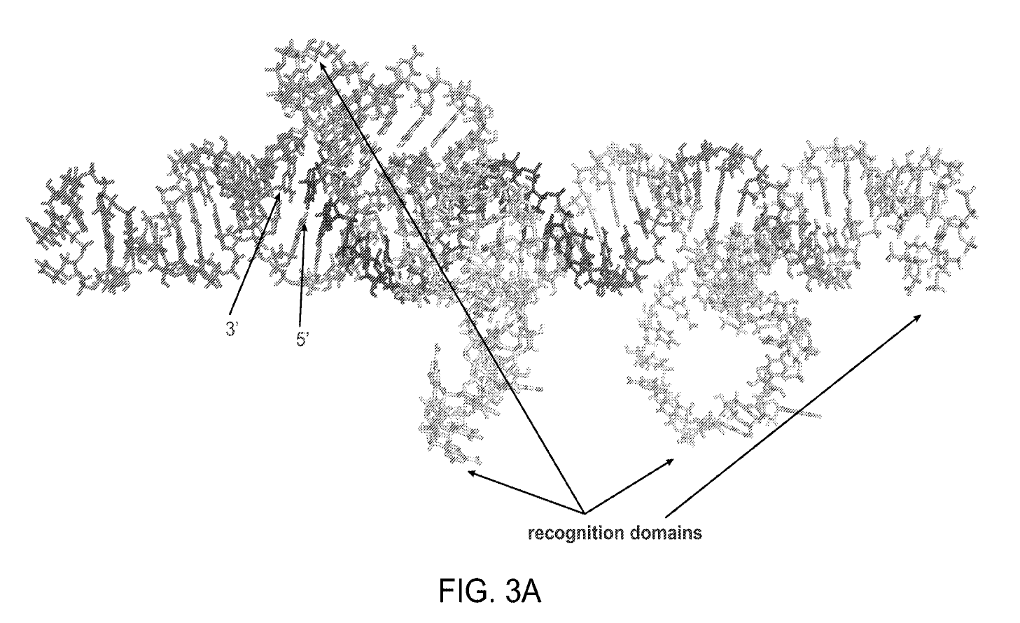

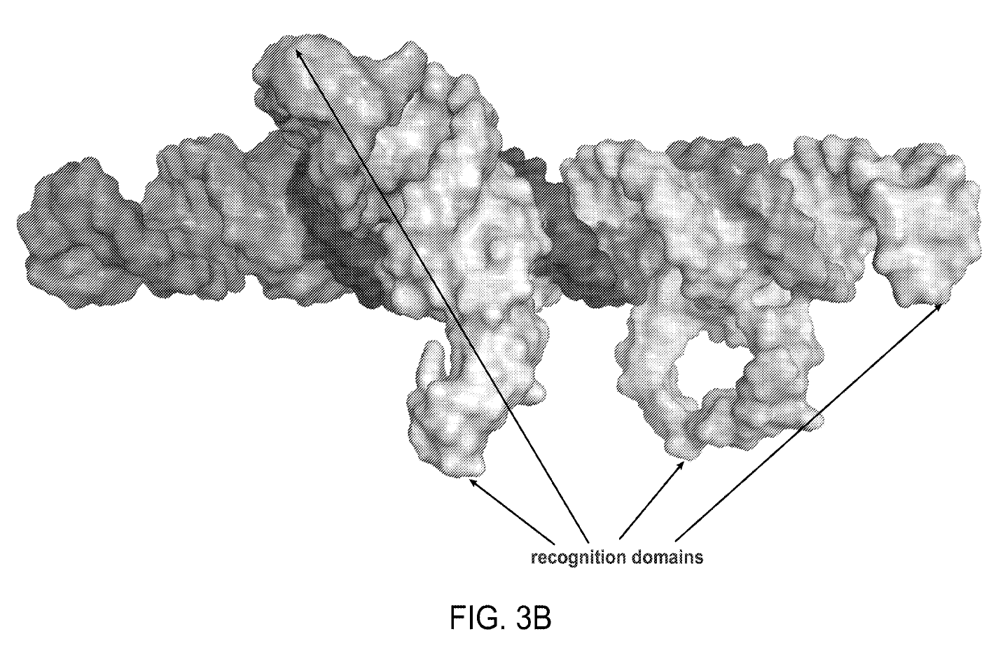

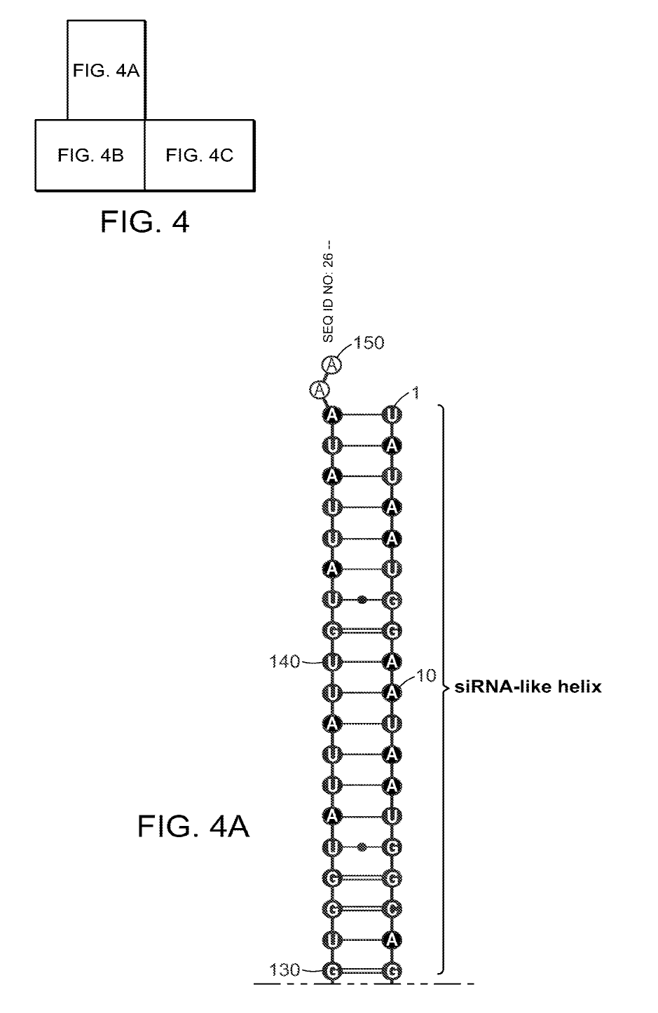

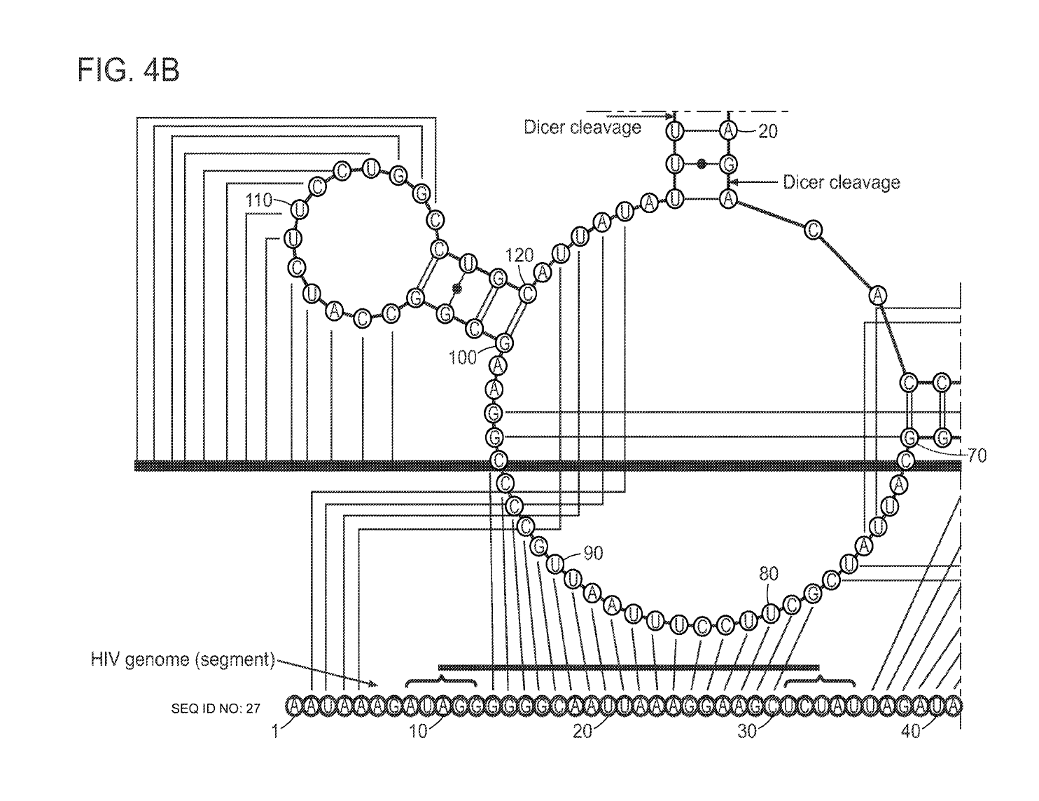

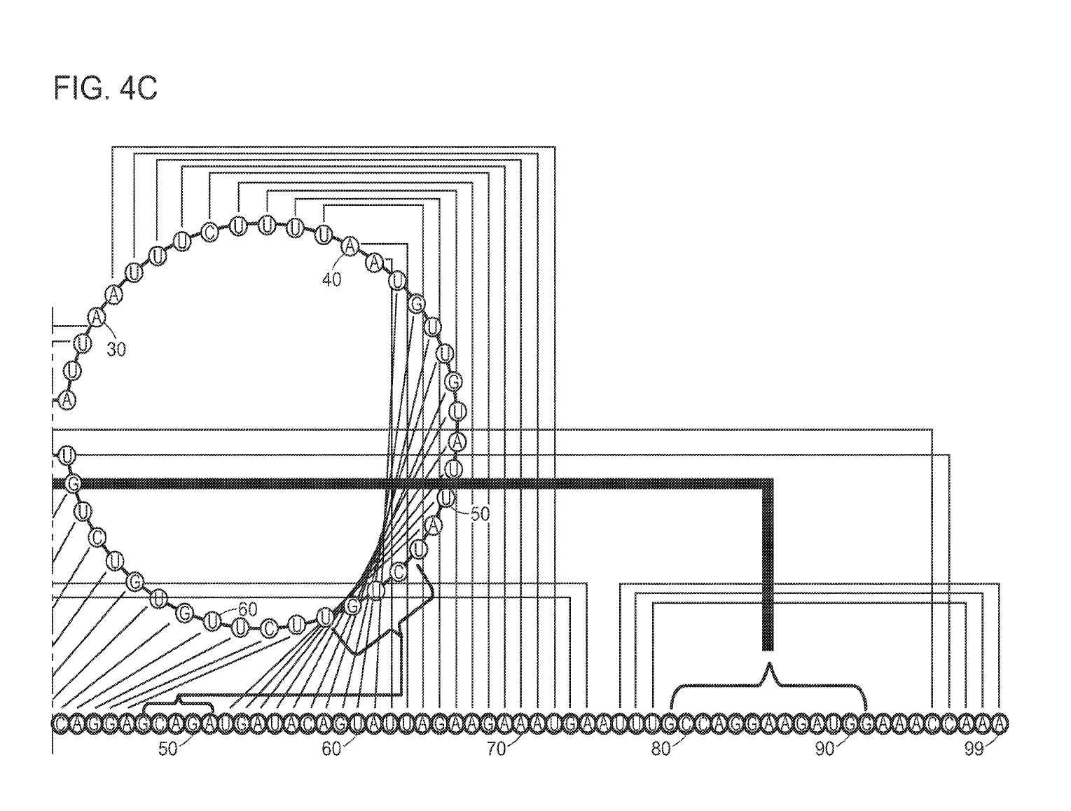

A diagram of the representative therapeutic switching RNA (called here tswRNA) is shown in FIG. 1. In the absence of the HIV RNA genome, the tswRNA is not in an active therapeutic conformation. The tswRNA is designed to possess one or more adjacent sequence regions that can bind to the viral RNA genome (see recognition domains in FIGS. 2, 3A, and 3B). The computational predictions show that the formation of the drug-virus complex will induce a conformational change in the synthetic tswRNA. The complex formed between the drug and viral genome exposes an siRNA-like hairpin FIG. 4. The human enzyme Dicer is able to cleave this hairpin structure, and pass one of its strands (the guide strand) to the RNA induced silencing complex (RISC), which in turn activates the RNA interference (RNAi) pathway. The guide strand is designed to be an antisense to a target RNA. The activation of the RNAi pathway will result in repression of the targeted RNA expression (e.g., mRNA or pathogenic RNA), thus increasing the likelihood of apoptosis of the infected cell.

In certain embodiments, several human apoptosis inhibitor genes (BCL-2, FLIP, STAT3 and XIAP) can be used as targets.

It is within the purview of the skilled artisan to modify the design of the trigger sequence and the target siRNA for various applications. Specifically, one can readily apply known trigger and siRNA sequences to the methods and designs described herein.

A library of different tswRNAs will be designed and synthesized with the goal to selectively kill HIV-infected cells.

In vitro and in vivo testing of a library of therapeutic switching RNAs targeting cancer, multiple subtypes of HIV, and RNA viruses in general. Initial in vitro experiments will be done in MDA-MB-231 cells containing the EGFP gene. The presence of the mRNA of this gene will trigger our designed switches to induce apoptosis in these cells. In one embodiment, the mRNA of the gene TWIST or CTGF will trigger the active conformation of the RNA switch, leading to the formation of an siRNA targeting the anti-apoptotic gene Survivin. In another embodiment, the mRNA of the gene TWIST or CTGF will trigger the active conformation of the RNA switch, leading to the formation of an siRNA targeting the mRNA of the EGFP gene. Part of the developed RNA construct may consist of modified nucleotides including DNA. Also, the construct can consist of more than one nucleotide sequence. Examples of potential initial recognition sites for HIV are Ldr-3 GGAGAGAGAUGGGUGCGAGTT position 782; Gag-5 GAAGAAAUGAUGACAGCAUTT position 1819; Pol-1 ACAGGAGCAGAUGAUACAMT position 2328; Pol-29 CAGUGCAGGGGAAAGAAUATT position 4811; Pol-47 GUGAAGGGGCAGUAGUAAUTT position 4963; R/T-5 AUGGCAGGAAGAAGCGGAGTT position 5969 with associated potential human anti-apoptotic genes (Bcl-2, FLIP, STAT3, XIAP, Survivin, etc.) as targets. A potential cancer recognition site is BCR-ABL with the previously mentioned anti-apoptotic genes as targets. A designed anti-HIV RNA switch that targets the mRNA of the BCL-2 anti-apoptotic gene is given by the sequence:

UAUAAUGCAAUAAUGCCACAGACACCAUUAAUUUCUUUUAAUGUUGUAUU
AUCUGUUCUUGUGUCUGUGGCAUUAUCGCUUCCUUUUAAUUGCCCCGGAA
GCGGCCAUCUUCCUGGCCUGCAUUAUAUUUGUGGUAUUAUUGUAUUAUAA
A.

As described herein, the siRNA targets can be pathogenic RNA. In addition, the siRNA targets include cancer related genes, for example—the hypoxia pathway: Hifl alpha, VEGF; DNA repair pathway: PARP; microRNAS: miR21, miR7, miR128a, miR210; cancer stem cells: genes in NOTCH, HEDGEHOG, PTEN, WNT, TGFbeta pathways; immune modulation: Interleukin (IL-6, IL-10) and genes in the JAK/STAT, SMAD, TNFalpha. In principle the concept can be expanded to include many genetically related diseases.

Computational Methods.

Several novel and leading bioinformatics approaches for RNA secondary structure prediction, RNA 3D structure modeling, and RNA sequence design are used to computationally design and test the therapeutic RNA switches. Computational results of a designed RNA switch indicate that it is inactive in its unbound state and active when bound to a trigger sequence. The resulting hairpin in the active tswRNA is an siRNA analog that targets the BCL-2 gene. Similar conditional RNA switches that target the remaining genes STAT3, FLIP and XIAP will be designed. In addition, three-dimensional models will be generated that will be subjected to molecular dynamics simulations. This will provide important information regarding the dynamic behavior of the synthetic RNA switches.

Experimental Methods.

The in silico designed library of tswRNAs will be tested in vitro and in HIV infected cells. First, all newly synthesized tswRNAs will be tested in vitro for their abilities to i) fold properly in the unbound state and ii) to be processed by human recombinant Dicer in the presence of complementary fragments of HIV mRNAs (indicating that the binding to the HIV RNA triggers the activation of tswRNAs through their switching into the therapeutically active state). Dicer activity should only be expected in cases where HIV RNA is present. If required, we will perform proper chemical modifications on tswRNA structures to promote Dicer processivity of the tswRNA in its active state. Next, HIV infected human cells (H9 and/or Jurkart cells) will be transfected with different types, combinations and concentrations of tswRNAs and apoptosis will be measured by flow cytometry using the BD™ MitoScreen (JC-1) flow cytometry kit. Non-infected cell lines will be used as a control. The next steps are the detailed characterization of the most promising therapeutic switching RNAs (mechanisms of switching, kinetics, thermodynamics, binding affinities, etc). We will apply lipid-based drug delivery approaches (the lab has expertise in the use of liposomes and bolaamphiphiles). This will make possible the further testing of the designed tswRNAs in different animal models with the long-term goal of human clinical trials. Lastly, we emphasize that we envision this to be a general approach that could be readily modified to develop potential functional cures for other RNA viruses.

### Example 2

### Two-Strand RNA Switch

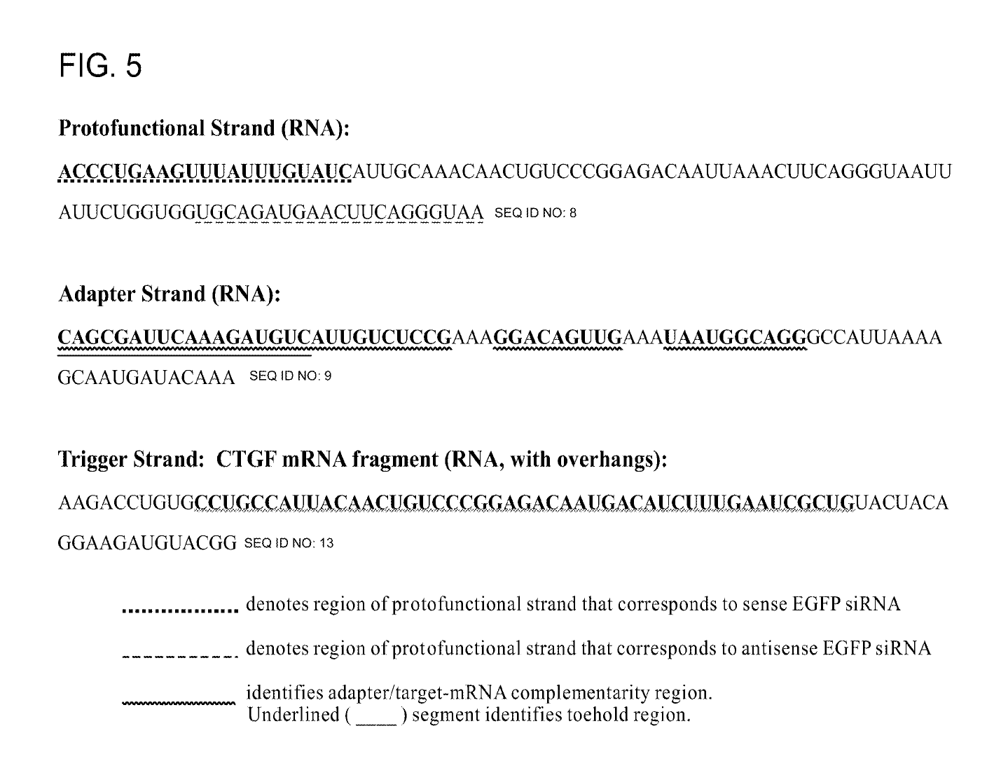

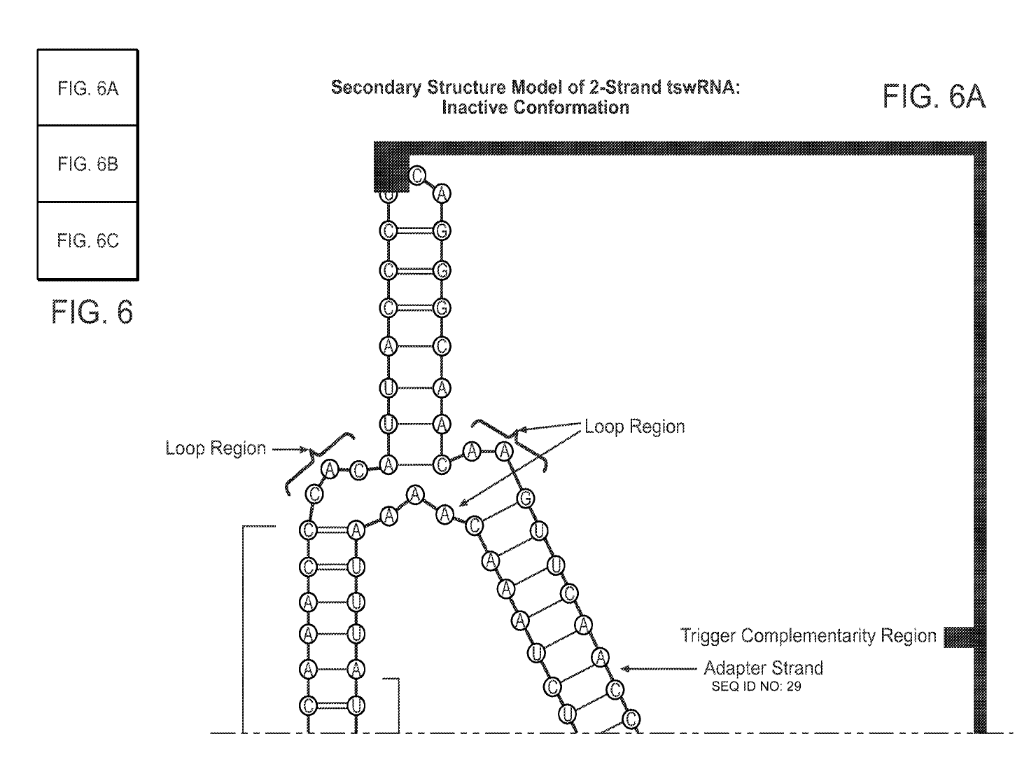

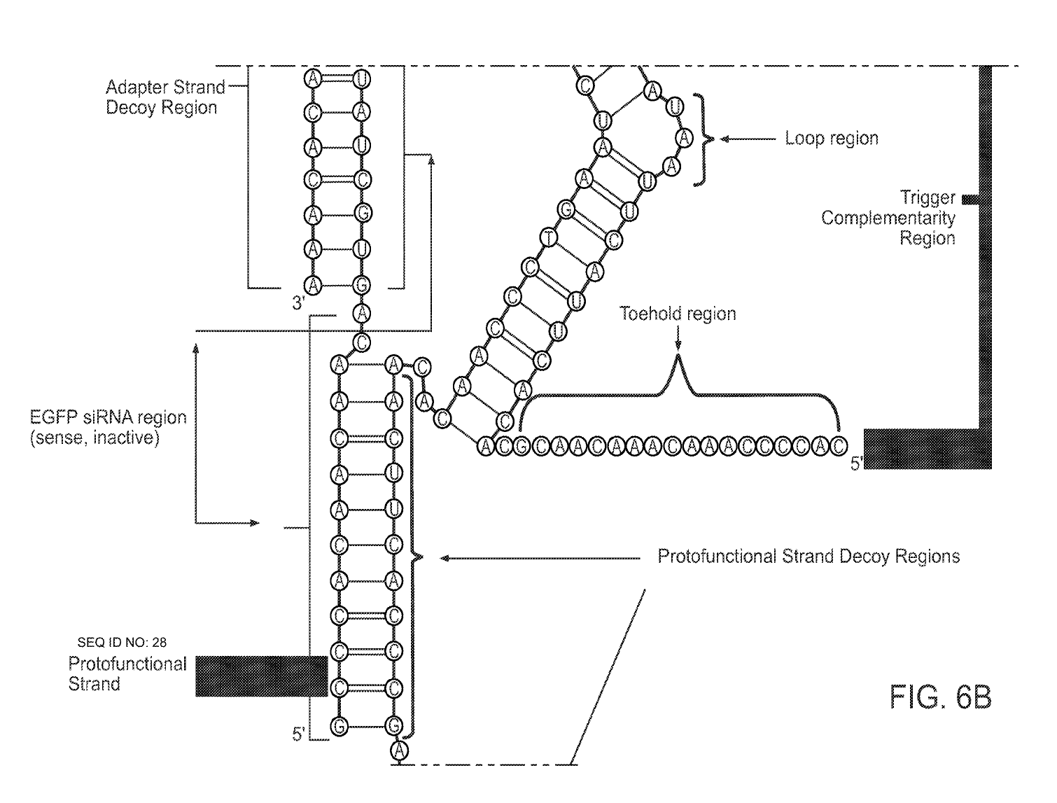

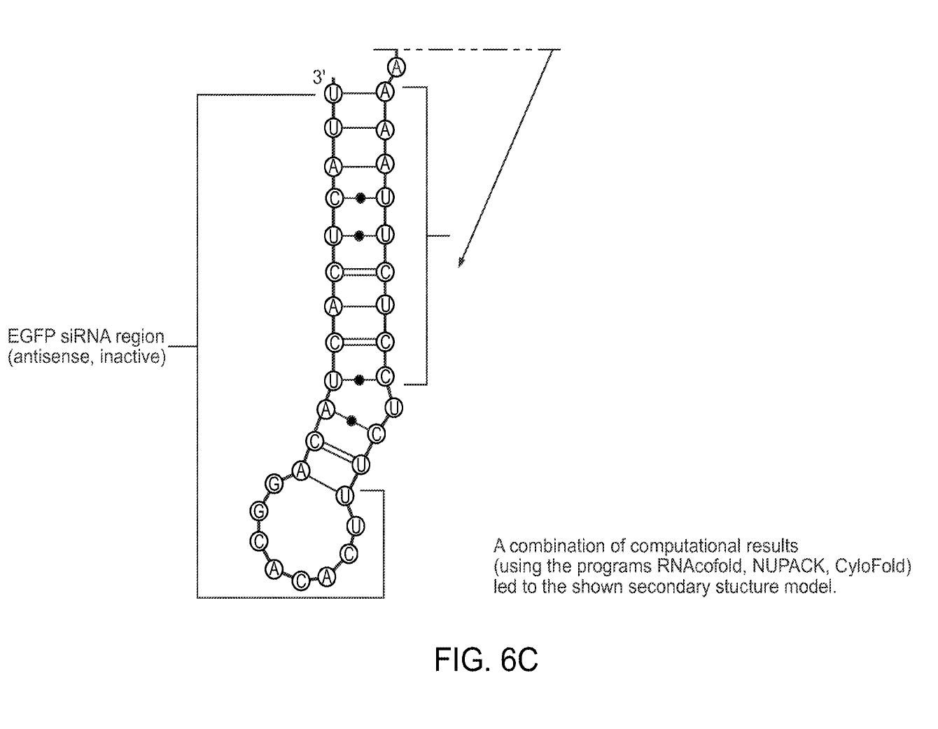

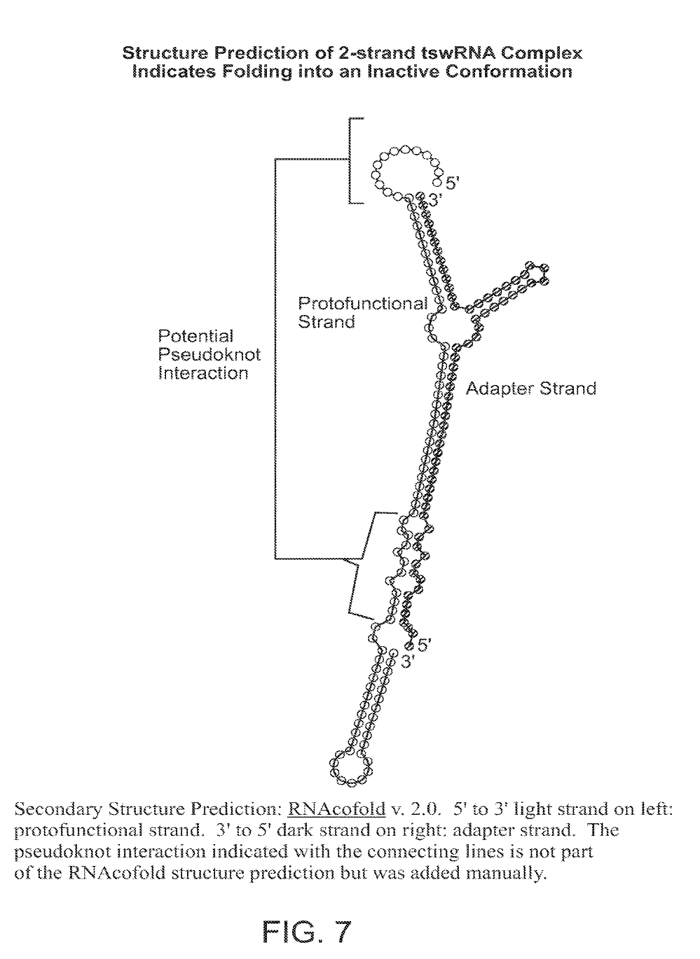

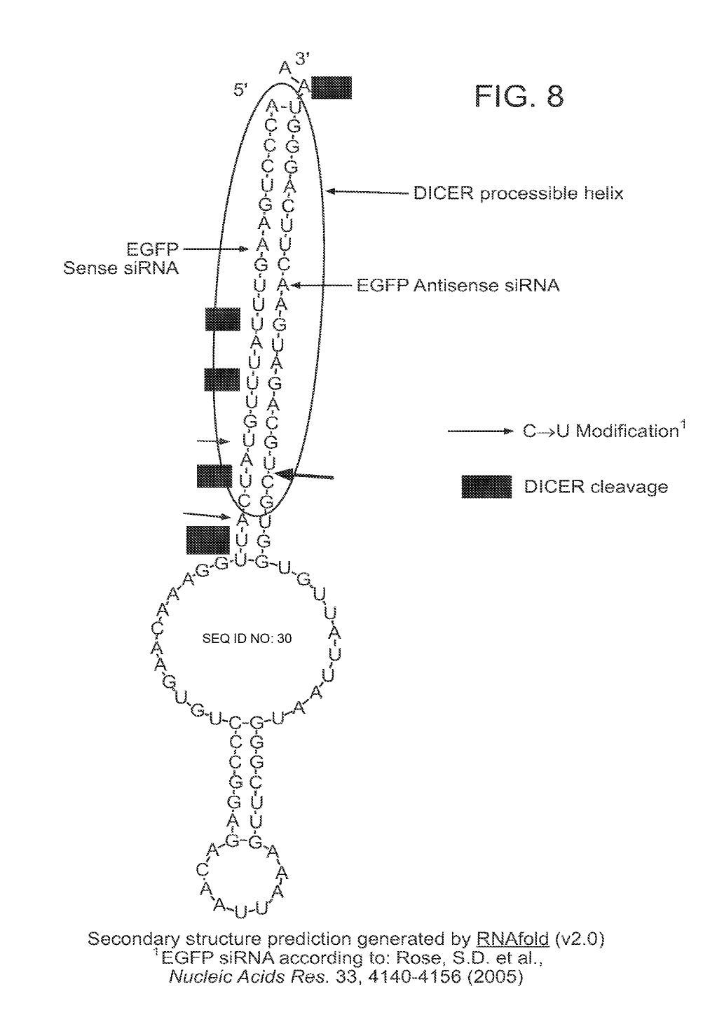

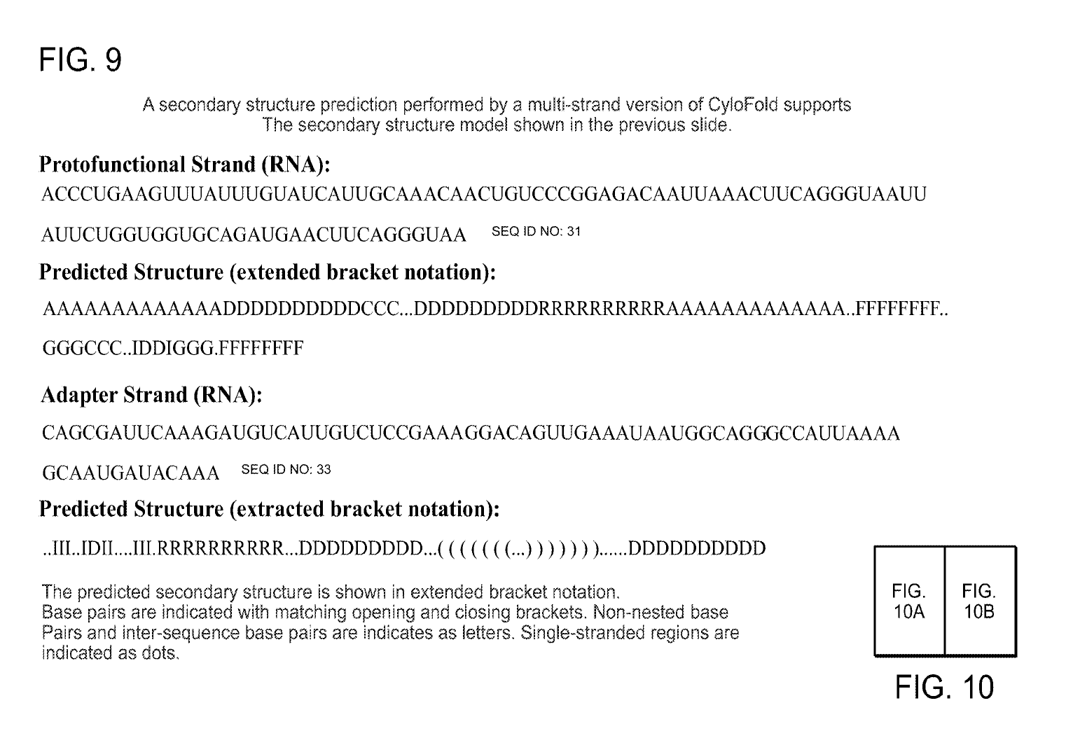

A tswRNA consisting of two nucleotide strands, an adapter strand and a protofunctional strand, is designed (FIG. 5). In the absence of a trigger sequence the two-strand tswRNA is in an inactive state (FIG. 6). However, this two-strand RNA-RNA (or alternatively RNA-DNA) complex is activated in the presence of a nucleotide sequence that acts as trigger and biomarker for the disease state (such as the presence of an differentially expressed mRNA) (FIGS. 7 & 8). In a non-limiting example, a region of the CTGF mRNA acts as trigger (the gene CTGF is highly expressed in a variety of cancers; CTGF is also known under the synonyms CCN2 or IGFBP8) (FIG. 9). Analogous designs could use as trigger nucleotide sequences regions of other RNAs that are differentially expressed in cancer cells (for example mRNAs of oncogenes) or pathogen-infected cells (for example viral genomic RNA or pathogenic mRNAs).

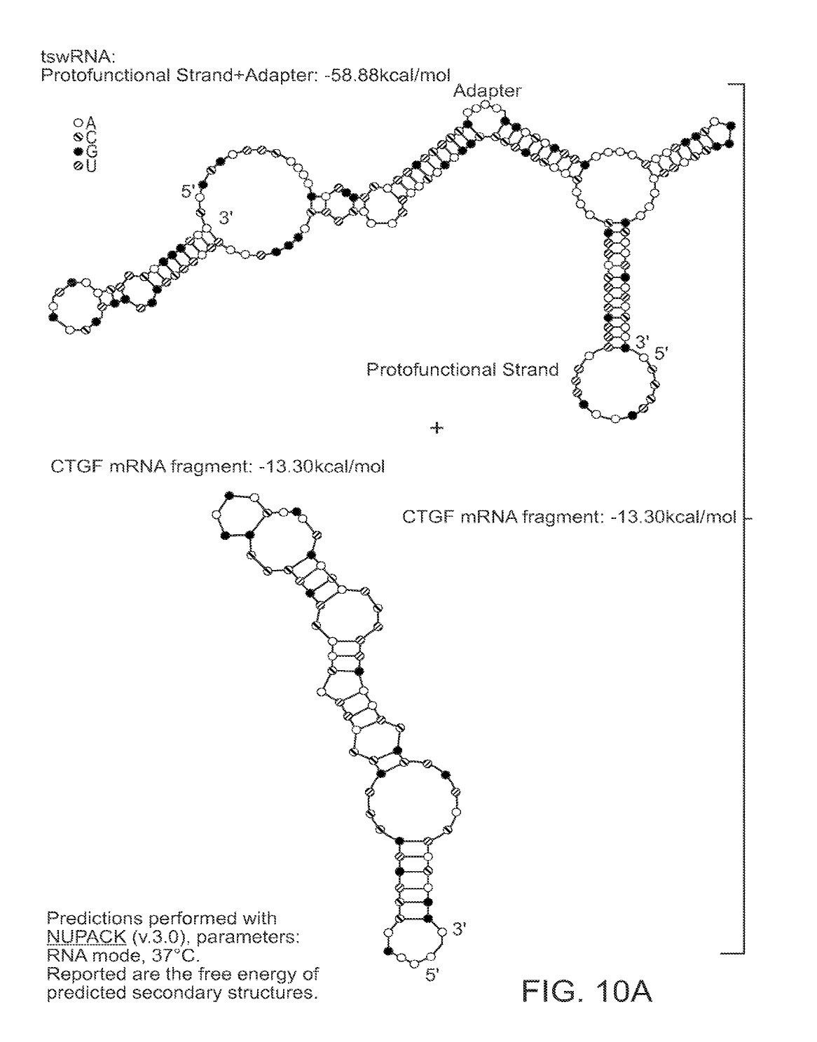

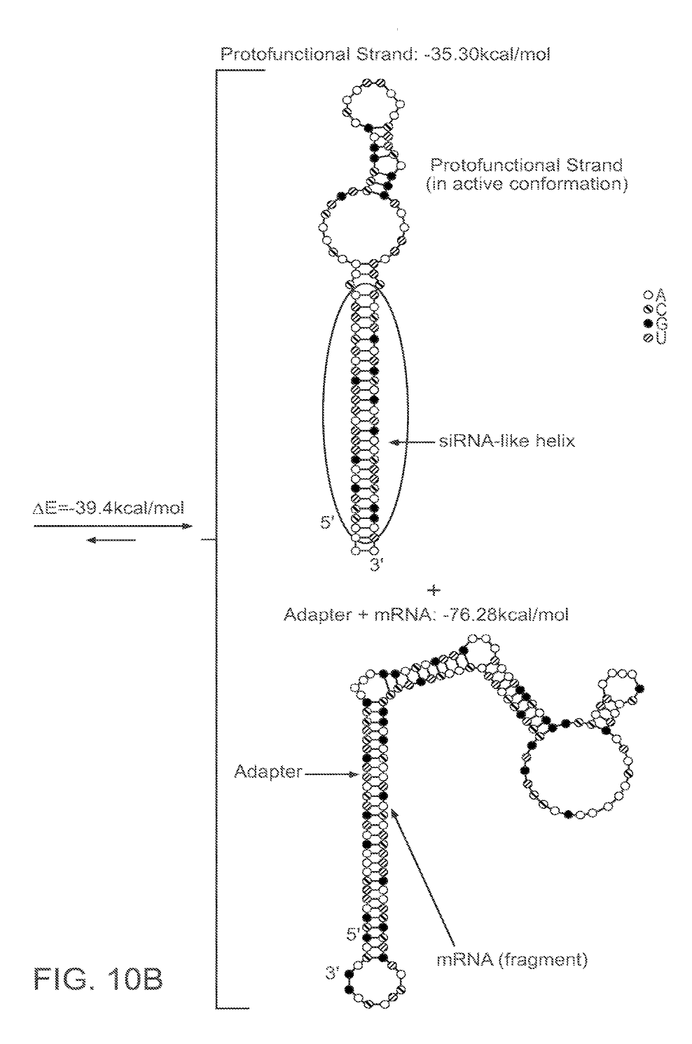

The down-regulation of the EGFP (enhanced GFP) gene will be used as an illustrative example to represent functionality in order to demonstrate the principle by using cell-lines that express CTGF (trigger sequence) and EGFP (target sequence). In other words, in the presence of the trigger sequence (the CTGF mRNA), the adapter binds to the trigger and the protofunctional strand of the tswRNA folds such that it exposes a DICER-processible siRNA-like helix that, when processed by DICER, targets and down-regulates the EGFP gene, thus leading to a decrease in detected fluorescence (FIG. 10). Instead of EGFP as a target gene, analogous designs could incorporate siRNA regions that target other genes such as apoptosis inhibitors (whose down-regulation leads to an increase in cell death) or other target sequences, the inhibition of which leads to a therapeutically beneficial effect. The target sequence could also represent non-coding RNA.

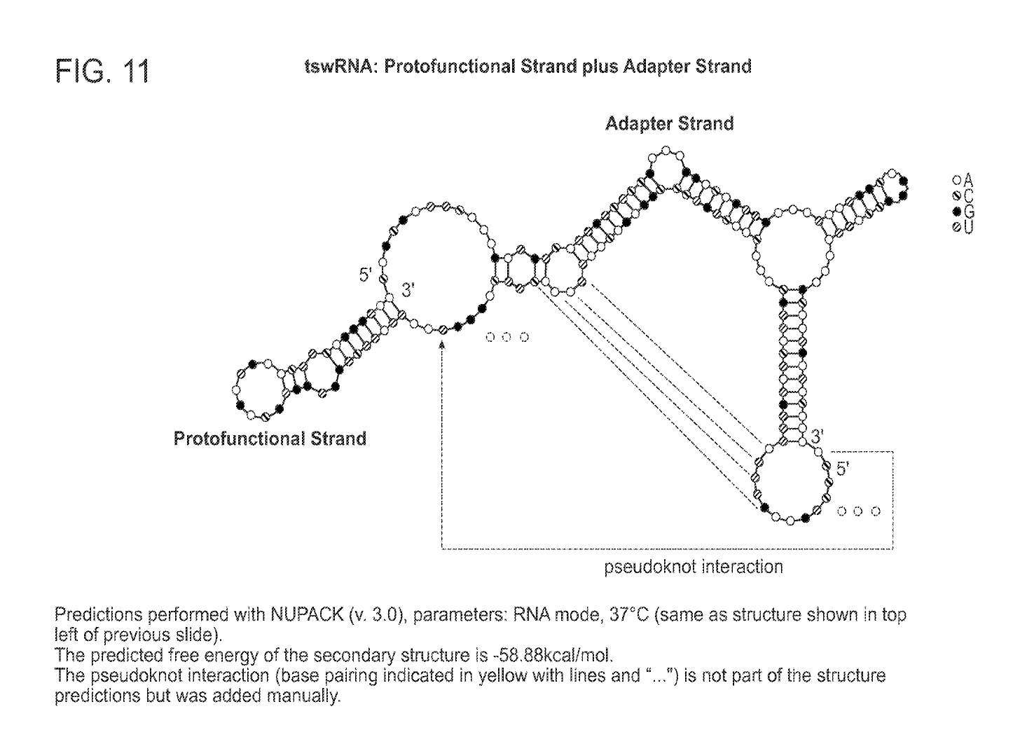

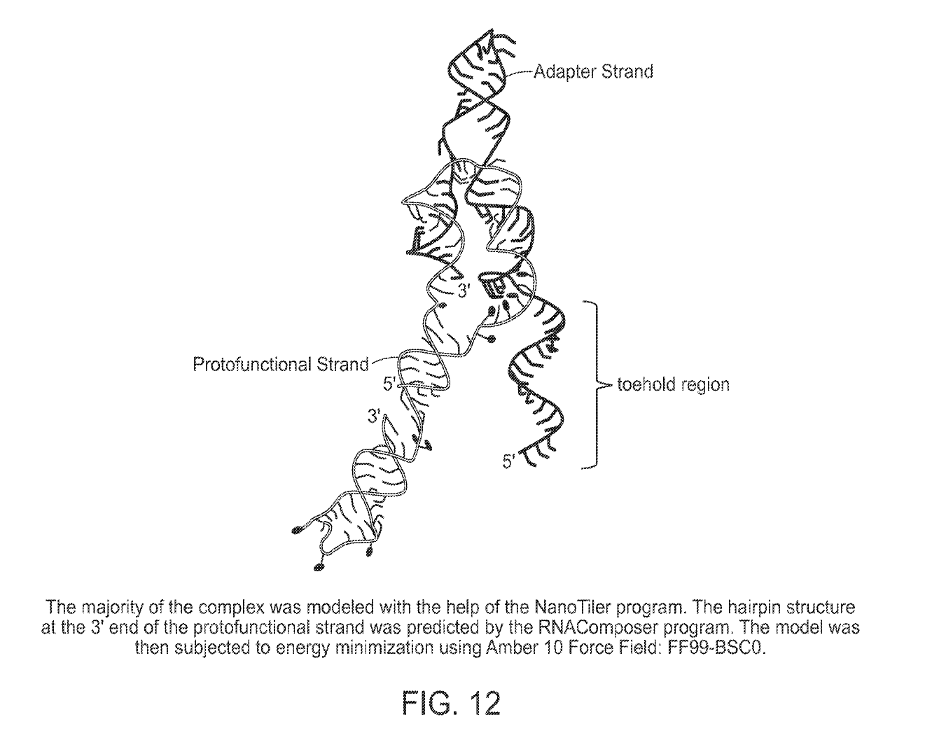

The two-strand design consists of a protofunctional RNA strand and an adapter strand that can be either RNA or DNA (FIGS. 11 and 12). The protofunctional strand folds (if not paired to another nucleotide strand) into a conformation that exposes a DICER-processible siRNA-like double helix (here: containing siRNA that targets EGFP).

The complex between the protofunctional strand and the adapter strand folds into a conformation that does not expose a DICER-processible siRNA-like helix. The protofunctional strand contains two sequence regions that, after DICER processing, correspond to a sense and an antisense siRNA. Those two siRNA regions are located at the 5′ end and the 3′ end of the protofunctional strand.

The inactive conformation (the complex between the protofunctional strand and the adapter strand) is stabilized with the help of several decoy regions, located on the adapter strand as well as the protofunctional strand that promote extensive base pairing between the adapter strand and the protofunctional strand and provide a structural alternative (decoy) compared to the stable siRNA duplex region.

The majority of the adapter strand is the reverse complement of the nucleotide strand that acts as a biomarker and trigger (for example, a region of the CTGF mRNA). Both the adapter strand and the protofunctional strand possess regions that correspond to single-stranded regions (loops) in the tswRNA complex in order to facilitate folding into the complex structure without steric clashes (FIG. 6—indicated as “loop regions”). The complex between the protofunctional strand and the adapter strand exposes a single-stranded toehold region (near the 5′ end of the adapter strand) that promotes the initiation of binding between the adapter strand and the trigger-RNA region. In analogous designs, one or several single-stranded toehold regions can be located at adapter strand regions other than the 5′ end.

Using a variety of computational tools (RNAfold, RNAcofold, NUPACK, CyloFold (multi-strand version), NanoTiler, RNAComposer) two RNA sequences corresponding to a two-strand twsRNA triggered by CTGF mRNA and targeting EGFP mRNA have been designed (FIG. 5).

Similar embodiments: the adapter strand could be DNA instead of RNA. Both protofunctional strand and adapter strand can contain a higher or smaller amount of loop regions and decoy regions arranged in orders that differ from those presented in the figures.

### Example 3

### Four-Strand RNA/DNA Hybrid Complex

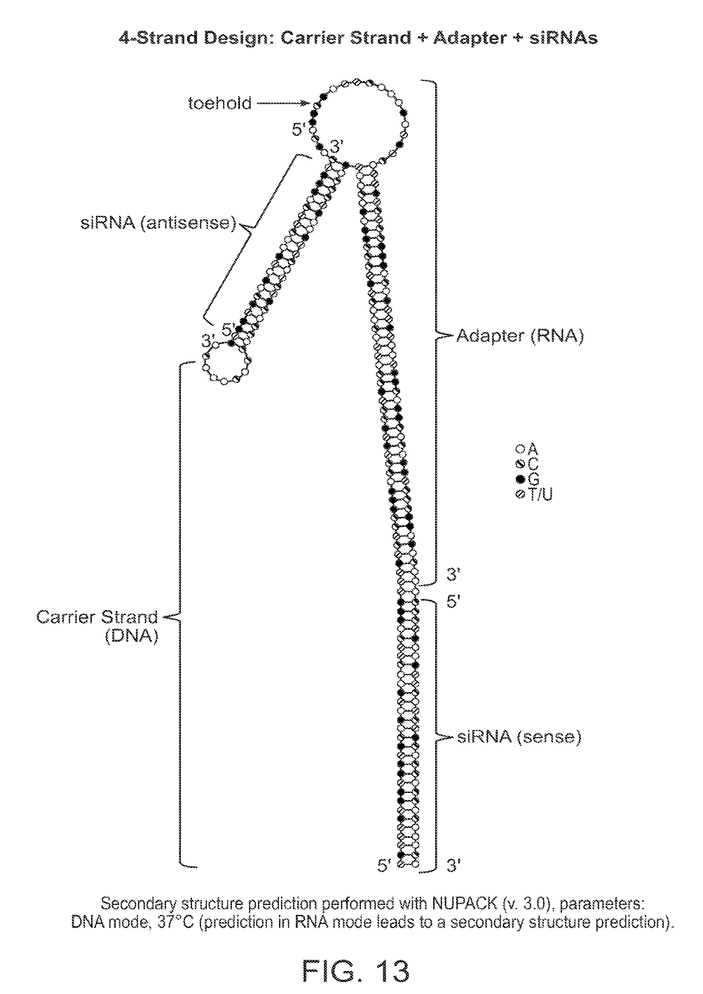

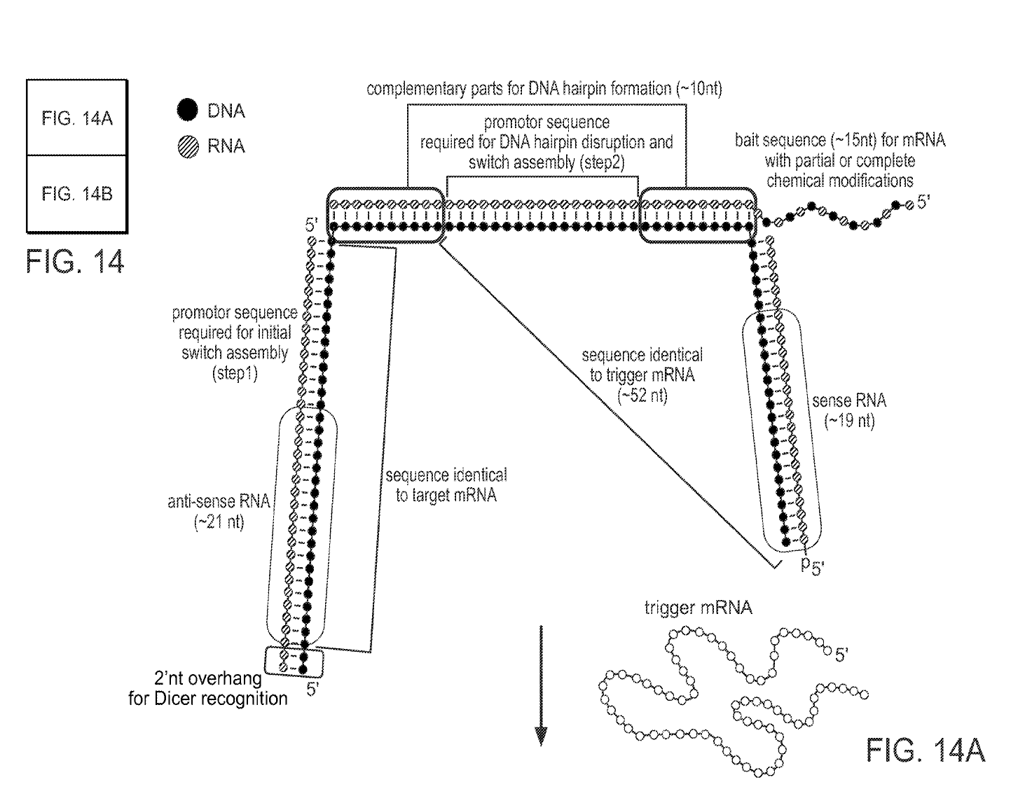

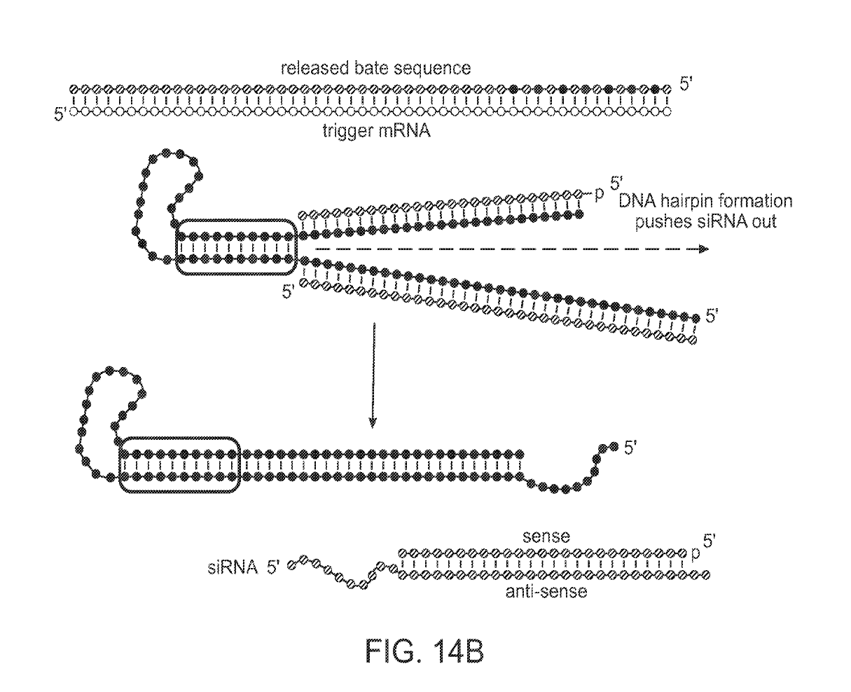

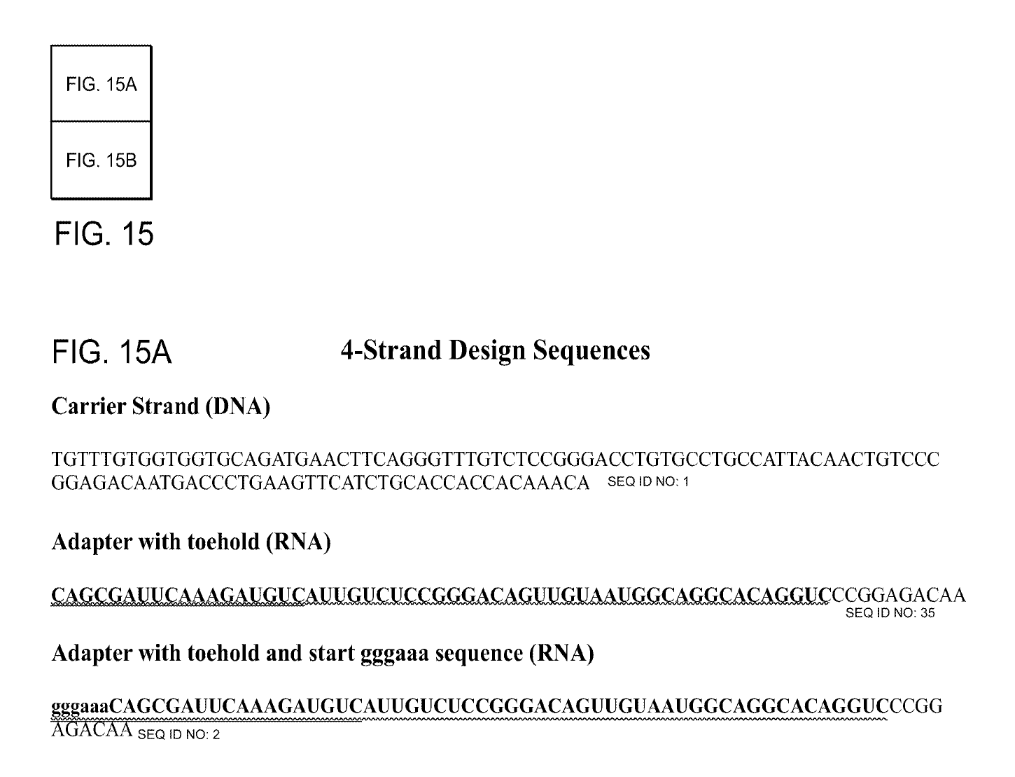

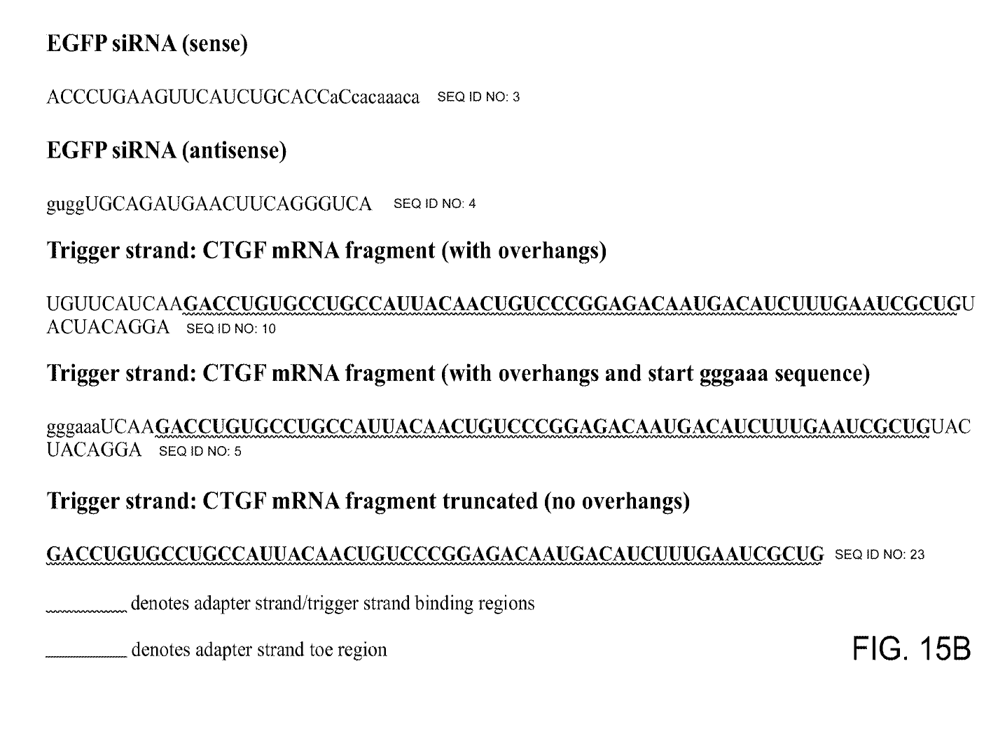

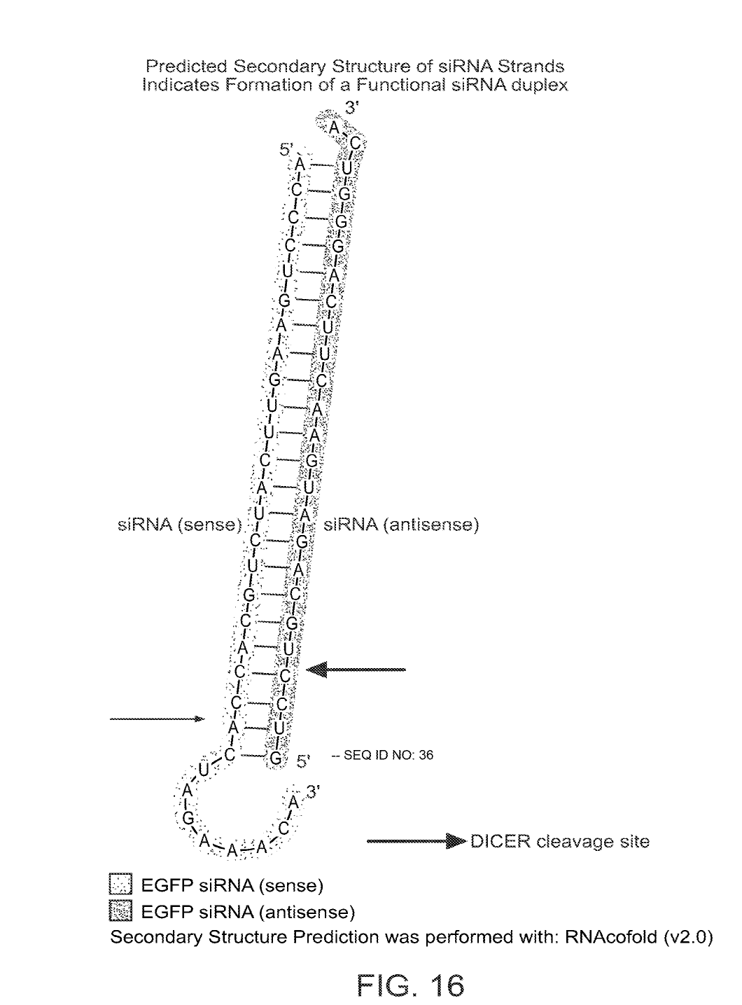

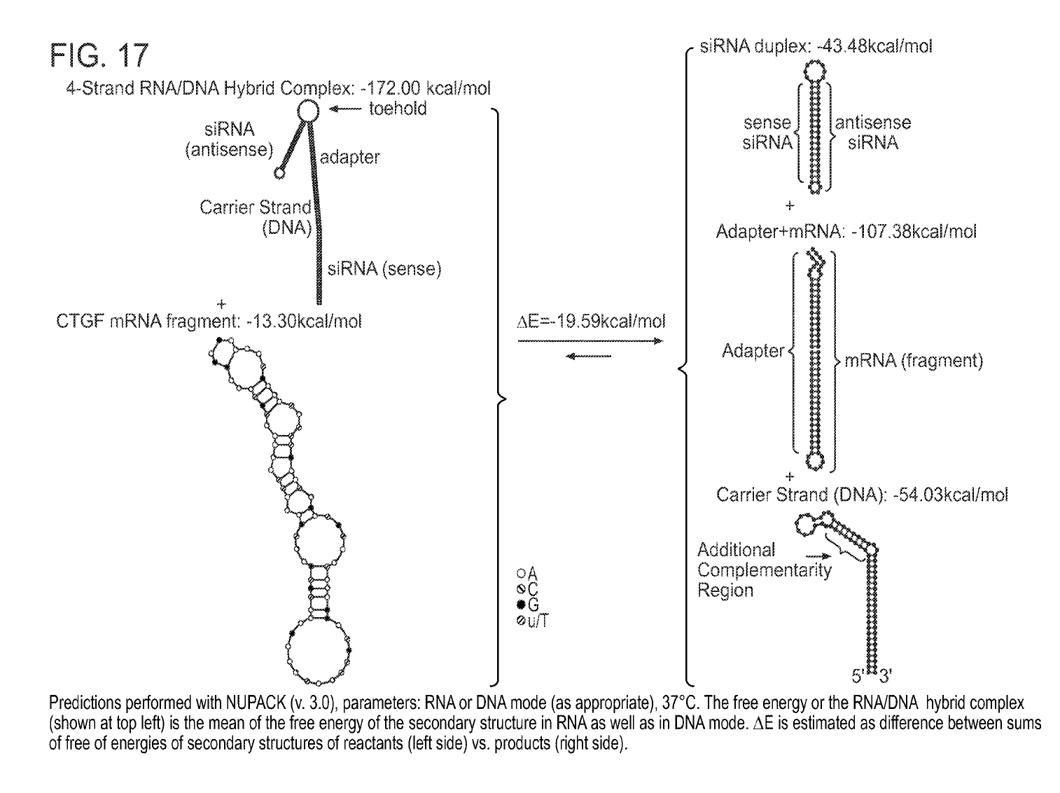

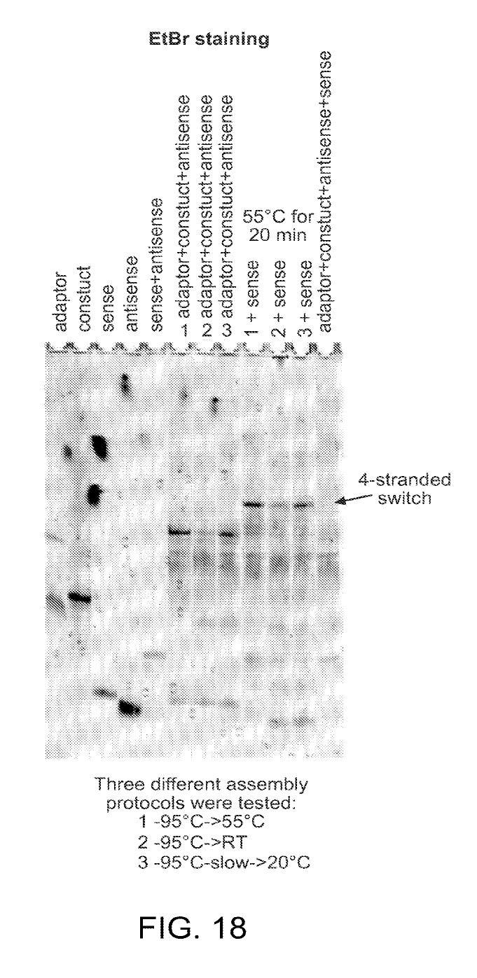

In another embodiment, the therapeutic nucleotide complex consists of three RNA strands (one sense siRNA, one antisense siRNA, and one adapter RNA) and one DNA strand (called the carrier strand) (FIGS. 13 and 18). The majority of the adapter strand is reverse-complementary to a nucleotide region strand that acts as trigger and biomarker (FIG. 14). In designing an illustrative example, a region of the CTGF mRNA was chosen to act as a trigger and siRNA strands were designed that would inhibit the expression of EGFP in a cell line (FIGS. 15 and 16). In the absence of a nucleotide trigger sequence, the therapeutic complex is in an inactive conformation (FIG. 17). In analogous designs, regions of other RNAs (such as mRNAs of oncogenes, viral genomic RNA) or DNAs could act as trigger sequences.

Binding of the adapter strand to a region of the trigger strand leads to a dissociation of the adapter strand from the therapeutic complex (FIGS. 14 &and 17). After binding of the adapter strand to the trigger strand (and dissociation of the adapter strand from the therapeutic complex), the remaining complex consisting of the carrier strand (DNA) and two siRNA strands changes conformation that leads to the formation of an siRNA duplex and a self-folding carrier strand. In another design, the adapter strand binds to the trigger nucleotide sequence, but does not completely dissociate from the carrier strand.

The carrier strand consists of several regions: a region that can bind to the sense siRNA, a region that can bind to the adapter strand and a region that can bind to the antisense siRNA (FIGS. 13 and 14). The carrier strand contains an additional complementarity region that promotes the formation of the siRNA duplex after removal of the adapter strand (FIG. 14). In other designs the carrier strand contains no additional complementarity region. In yet other designs, the carrier strand contains several additional complementarity regions.

The siRNA duplex (that forms as a result of the binding of the trigger strand to the adapter strand), is recognized and processed by DICER, thus leading to the activation of the RNA silencing pathway (FIG. 14). The activation of the RNA silencing pathway leads to the down-regulation of the desired target gene or pathway. In the design illustrated in FIGS. 15-17, the target gene is EGFP. Analogous designs (utilizing other siRNAs) could target mRNAs of other genes such as anti-apoptotic genes, oncogenes, cytokines or viral genes. In another embodiment, the targeted gene product is a noncoding RNA instead of an mRNA.

It is within the purview of the skilled artisan to incorporate different types of siRNAs and different types of trigger sequences. Changing the siRNAs in the four-strand design does not affect the adapter strand sequence. Only the parts of the construct/carrier strand base pairing with the siRNAs have to be modified to facilitate base pairing with the siRNA strands. Changing the trigger sequence results in a change of the adapter strand (such that it base pairs with the trigger sequence) and a change in the part of the construct/carrier strand such that it base pairs with the adapter strands.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A therapeutic RNA switch comprising:
at least one polynucleotide sequence that can bind to a trigger sequence; and
an antisense oligonucleotide and a sense oligonucleotide in which the antisense oligonucleotide is complementary to a target RNA, wherein the RNA switch can switch between an inactive state and an active state in the presence of the trigger sequence.

2. The therapeutic RNA switch of claim 1, wherein in the inactive state no partially formed siRNA-like helices exist.

3. The therapeutic RNA switch of claims 1, wherein the therapeutic RNA switch undergoes a conformational change in the presence of the trigger sequence which causes the antisense and sense oligonucleotides to form an siRNA-like helix.

4. The therapeutic RNA switch of claim 3, wherein the siRNA-like molecule reduces or inhibits the target RNA.

5. A two-strand therapeutic RNA switch comprising:
a complex between an adapter polynucleotide strand and a protofunctional polynucleotide strand, wherein the adapter polynucleotide can bind a trigger sequence and the protofunctional polynucleotide strand forms an siRNA-like RNA double helix when the adapter polynucleotide strand binds the trigger sequence.

6. The two-strand therapeutic RNA switch of claim 5, wherein the siRNA-like RNA double helix comprises an antisense oligonucleotide and a sense oligonucleotide in which the antisense oligonucleotide is complementary to a target RNA.

7. The two-strand therapeutic RNA switch of claim 5, wherein in the absence of a trigger sequence no partially formed siRNA-like helices exist.

8. The two-strand therapeutic RNA switch of claim 5, wherein the siRNA-like RNA double helix reduces or inhibits the target RNA.

9. A four-strand therapeutic RNA/DNA hybrid complex comprising:
a complex between a DNA carrier polynucleotide strand, an RNA adapter polynucleotide strand, a sense siRNA strand, and an antisense siRNA strand, wherein binding of the RNA adapter polynucleotide strand to a trigger sequence removes the RNA adapter strand from the complex and results in a conformational change wherein the sense siRNA strand and the antisense siRNA strand form an siRNA duplex.

10. The four-strand therapeutic RNA/DNA hybrid complex of claim 9, wherein in the absence of a trigger sequence no partially formed siRNA-like helices exist.

11. The four-strand therapeutic RNA/DNA hybrid complex of claim 9, wherein the four-strand therapeutic RNA/DNA hybrid complex undergoes a conformational change in the presence of the trigger sequence which causes the antisense and sense oligonucleotides to form an siRNA-like helix.

12. The four-strand therapeutic RNA/DNA hybrid complex of claim 9, wherein the siRNA-like molecule reduces or inhibits the target RNA.

13. The therapeutic of claim 1, wherein the trigger sequence is a nucleic acid present in a targeted cell of interest.

14-30. (canceled)

31. The therapeutic of any one of claims 1-30, further comprising a recognition domain, functional moiety, or aptamer.

32-35. (canceled)

36. A method of inhibiting or reducing the expression of a target gene in a cell comprising contacting the cell with a therapeutically effective amount of the therapeutic of claim 1.

37. A method of killing a pathogen infected cell comprising contacting the cell with a therapeutically effective amount of the therapeutic of claim 1.

38. A method of inhibiting replication of a pathogen in a cell comprising contacting the cell with a therapeutically effective amount of the therapeutic of claim 1.

39. The method of claim 36, wherein the cell is in a subject.

40. A method of reducing pathogenic burden in a subject comprising administering a therapeutically effective amount of the therapeutic of claim 1 to the subject.

41. The method of claim 40, wherein the subject is at risk of developing a pathogenic infection.

42. The method of claim 40, wherein the subject is diagnosed with having a pathogenic infection.

43. A method of treating or preventing a pathogenic infection in a subject comprising administering a therapeutically effective amount of the therapeutic of claim 1 to the subject.

44. The method of claim 43, wherein the method reduces the pathogenic burden, thereby treating or preventing the pathogenic infection.

45. The method of claim 43, wherein the method induces death in infected cell, thereby treating or preventing the pathogenic infection.

46. The method of claim 39, wherein the subject is a mammal.

47. The method of claim 46, wherein the subject is a human.

48. The method of claim 37, wherein the pathogen is a virus, bacteria, fungus, or parasite.

49-51. (canceled)

52. The method of claim 37, wherein the method further comprises contacting the cell with a therapeutically effective amount of a second therapeutic agent or administering a therapeutically effective amount of the second therapeutic agent to the subject.

53-55. (canceled)

56. A method of killing a neoplastic cell comprising contacting the cancer cell with a therapeutically effective amount of the therapeutic of claim 1.

57. A method of treating a subject having a neoplasia, the method comprising administering to a subject a therapeutically effective amount of a therapeutic of claim 1, thereby treating the subject.

58. The method of claim 56, wherein the neoplastic cell is a cancer cell which is present in a solid tumor.

59-61. (canceled)

62. A composition comprising a therapeutic claim 1.

63. A pharmaceutical composition comprising a therapeutic of claim 1.

64. The pharmaceutical composition of claim 63 further comprising a pharmaceutically acceptable excipient, carrier, or diluent.

65-74. (canceled)

75. A kit comprising a therapeutic of claim 1.

76-91. (canceled)
